(19) United States
(12) Patent Application Publication
(10) Pub. No.: US 2026/0295050 A1
(43) Pub. Date: Oct. 1, 2026

(54) PHOTOACTIVATABLE NANOPARTICLES AND USES THEREOF AS AGENTS FOR IMMUNOTHERAPY COMBINATIONS

(71) Applicant: Board of Regents, The University of Texas System, Austin, TX (US)

(72) Inventors: Girgis Obaid, Allen, TX (US); Nimit Shah, Dallas, TX (US); Nikita Kulkarni, Richardson, TX (US)

(73) Assignee: Board of Regents, The University of Texas System, Austin, TX (US)

(21) Appl. No.: 19/578,190

(22) Filed: Mar. 25, 2026

Related U.S. Application Data
(60) Provisional application No. 63/777,219, filed on Mar. 25, 2025.

(51) Int. Cl. A61K 41/00; A61K 35/17; A61K 40/31; A61K 47/54; A61P 35/00; G16C 20/10

(52) U.S. Cl. CPC A61K 41/0071; A61K 35/17; A61K 40/31; A61K 47/543; A61P 35/00; G16C 20/10; A61K 2121/00

(57) ABSTRACT

Lipid nanoparticles described herein are useful in photodynamic therapy (PDT), a light-activated cancer treatment that uses photosensitizers (PSs) to generate reactive oxygen species (ROS), leading to tumor cell death. These lipid nanoparticles are capable of producing varying levels of oxygen-based radicals and singlet oxygen. In preferred embodiments, the lipid nanoparticles are verteporfin-lipid nanoparticles. Fine-tuning design features of the lipid nanoparticles, such as Type I ROS generation and predominantly ER and mitochondrial localization, provides a more robust induction of immunogenic cell death (ICD).

This application claims priority to U.S. Provisional Patent App. Ser. No. 63/777,219, entitled “Photoactivatable Nanoparticles and Uses Thereof as Agents for Immunotherapy Combinations,” filed on Mar. 25, 2025, the entire contents of which are hereby incorporated by reference.

This invention was made with government support under Grant Nos. R00CA215301 and R01EB034360 awarded by the National Institutes of Health. The government has certain rights in the invention.

### BACKGROUND

Immunotherapy has emerged as a powerful strategy for managing various cancers, including solid tumors. Immunotherapy regimens are approved for a number of cancers and are being evaluated as part of combination treatments for multiple difficult-to-treat solid tumors, such as pancreatic ductal adenocarcinoma (PDAC), in clinical trials. Commonly used immune checkpoint inhibitors (ICIs) approved for use in the clinic, and also being evaluated in clinical trials for several cancers, include antibodies targeting programmed death protein-1 (α-PD-1), programmed death ligand-1 (α-PD-L1), and cytotoxic T-lymphocyte-associated protein 4 (α-CTLA-4). These inhibitors function by blocking immune checkpoints that normally suppress T-cell education and activity, thereby restoring the immune system's ability to recognize and attack tumors. Despite the clinical success of ICIs, the majority of cancer patients do not respond well to ICI therapy. A key challenge is treatment resistance, driven by the accumulation of heterogeneous immunosuppressive cell populations in the tumor microenvironment (TME), some of which directly suppress T-cell responses which limits the effectiveness of ICIs. Therefore, novel combinatorial strategies are needed to both counteract the immunosuppressive TME and enhance immune activation against cancer. Notably, combining some photodynamic therapy (PDT) protocols with ICI has been shown to enhance systemic antitumor immunity in pre-clinical animal models.

Photodynamic therapy (PDT) is a light-activated cancer treatment that uses photosensitizers (PSs) to generate reactive oxygen species (ROS), leading to tumor cell death. In some cases, PDT, can induce tumor immune enhancement, but not always. It is not understood how or why some types of PSs and PS formulations can induce tumor immune enhancement and some cannot.

PDT involves the activation of PSs using light to generate ROS, which can be phototoxic, immunomodulatory, or biomodulatory, depending on the photosensitizer concentration and the light dose. PDT-induced cell death is influenced by multiple factors including ROS production, subcellular localization of the photosensitizer and its concentration, and the light dose. Traditionally, necrosis, autophagy, and apoptosis have been recognized as the primary cell death pathways following PDT. However, recent studies have identified several non-conventional pathways such as ferroptosis, pyroptosis, and paraptosis. Further, immunogenic cell death (ICD) can result from these pathways, and PDT is considered one of the most potent inducers of ICD. Post-PDT, stressed or dying cells release or translocate damage-associated molecular patterns (DAMPs) such as calreticulin, heat shock protein (HSP)-60, HSP-70, high mobility group box 1 (HMGB1), and extracellular ATP. Further, these DAMPs facilitate the recruitment of antigen-presenting cells like macrophages and dendritic cells which initiate a cascade of cellular responses that improve therapeutic outcomes by activating a T-cell adaptive response and by generating immune memory. Hypericin-PDT can induce ICD by examining the exposure of ICD markers calreticulin and HSP-70 in cancer cells. PDT can induce ICD through the release of DAMPs and can increase the number of neutrophils and macrophages post-PDT with the photosensitizer 5-ALA in a mouse model of actinic keratosis. Further, PDT has been used to induce ICD in a mouse colorectal cancer model using the photosensitizer 5-ALA, while a combination of verteporfin (benzoporphyrin derivative; BPD) and doxorubicin has been employed to induce ICD in vivo. Triple-targeted liposomal BPD has been used to induce ICD by assessing levels of calreticulin, HSP-60, HSP-70, and HMGB1 in a 3D model of pancreatic cancer. The liposomal formulation of BPD can induce ICD using sub-ablative photodynamic priming in mice bearing head and neck cancer.

Similarly, low, non-curative PDT doses have been suggested to be important for anti-tumor immune responses in vivo, while efficient T-cell responses and abscopal effects have been reported using high, curative PDT doses. Meanwhile, the impact of the type of photochemically generated ROS and subcellular localization of photosensitizer on ICD has not been sufficiently studied. Various reports in the literature claim that endoplasmic reticulum localization of photosensitizers is important for ICD, while others demonstrate efficient ICD using photosensitizers that exclusively localize in the lysosomes. However, no systematic and comprehensive study has investigated the interrelationship between the type of photochemical reactions, the subcellular localization, and the degree of phototoxicity on the efficacy of inducing ICD.

### SUMMARY

The present disclosure relates to photoactivatable nanoparticles and their uses in cancer therapy and treatment.

Lipid nanoparticles and liposomes are safe and approved formulations for drug delivery. Notably, the FDA-approved liposomal formulation of verteporfin (Visudyne®) has demonstrated tumor necrosis in pancreatic cancer patients, highlighting the potential of lipid-based photosensitizer (PS) delivery systems. However, the PS readily leaks from Visudyne®, and it is not known how or why some conditions for Visudyne PDT can induce tumor immune enhancement and some cannot. Preferred embodiments disclosed herein relate to formulations that address the leakiness of the PS by utilizing lipid conjugates of it and uses a machine learning approach to optimize the formulations for inducing tumor immune enhancement.

This disclosure relates to lipid nanoparticles for varying ROS production. These lipid nanoparticles are capable of producing varying levels of oxygen-based radicals and singlet oxygen. In preferred embodiments, the lipid nanoparticles are verteporfin-lipid nanoparticles such as liposome Lipo 20:0 BPD-PC, which generates high levels of singlet oxygen, and lipid nanoparticle LNP BPD-Cholesterol, which produces high levels of hydroxyl radicals and peroxynitrite anions. A shift from Type II to Type I photochemistry was observed in the solid lipid nanoparticle formulation of verteporfin, due to the presence of ionizable SM-102, which impacts ROS generation and PDT efficacy. Increased Type I ROS production is strongly correlated with higher HMGB1 and HSP-70 expression.

This disclosure also relates to activation of innate and adaptive immunity via immunogenic cell death (ICD). Verteporfin-lipid nanoparticles, when activated with sub-therapeutic doses, induce oxidative stress in cancer cells, triggering the release of key damage-associated molecular patterns (DAMPs) such as calreticulin, HMGB1, and HSP-70. These DAMPs serve as immunostimulatory signals recognized by antigen-presenting cells (APCs), particularly dendritic cells. Upon uptake, dendritic cells migrate to lymph nodes, where they present tumor-associated antigens to naïve T cells, leading to their activation and differentiation into tumor-specific cytotoxic T lymphocytes (CTLs). This cascade enhances adaptive immune responses, promoting long-term tumor surveillance and eradication. Therefore, photodynamic therapy using LNP-based formulations can facilitate ICD-driven T cell priming, making them an advantageous platform for cancer immunotherapy.

This disclosure also relates to tunable subcellular targeting of lipid nanoparticles for enhanced ICD. This disclosure demonstrates the engineering of verteporfin-lipid nanoparticles with tunable subcellular localization to enhance ICD. Preferred embodiments including Lipo 20:0 BPD-PC, LNP 20:0 BPD-PC, and LNP 16:0 BPD-PC, which primarily localized within lysosomes, whereas preferred embodiment LNP BPD-Cholesterol exhibited dual localization in both lysosomes and mitochondria. Notably, preferred embodiment LNP Free BPD demonstrated the highest accumulation within the endoplasmic reticulum (ER) and mitochondria. These findings indicate that lipid conjugation of verteporfin significantly influences intracellular distribution, which may, in turn, affect photodynamic efficiency. The verteporfin-lipid nanoparticles that showed the highest localization in mitochondria and the ER also exhibited increased HMGB1 and HSP-70 expression, suggesting their critical role in regulating ICD. This indicates that subcellular targeting plays a crucial role in determining PDT efficacy and immune activation, with mitochondrial and ER localization potentially enhancing ICD induction.

Immunogenic cell death (ICD) is the first step of adaptive immune responses against cancers that can be elicited by photodynamic therapy (PDT). The mechanisms underlying PDT-induced ICD are multi-faceted and remain understudied, especially for lipid nanoparticle formulations of photosensitizers. Reports on the importance of the type of photochemically-generated reactive oxygen species (ROS), whether Type I species (i.e. radicals) or Type II species (i.e. singlet oxygen), subcellular photosensitizer localization, and the extent of cellular photodamage for PDT-induced ICD are mixed. This disclosure includes the first systematic multi-variate and data-driven study of the contribution of the type of photochemical ROS produced, the subcellular localization of the photosensitizer, and the extent of cellular photodamage on PDT-induced ICD. PDT was performed in murine pancreatic cancer cells using 690 nm light activation of a panel of verteporfin-lipid nanoparticles (V-LNPs) containing unmodified verteporfin (benzoporphyrin derivative; BPD) or verteporfin conjugated to cholesterol, 20:0 lyso PC, or 16:0 lyso PC. Using an unsupervised machine learning model, namely, Principal Component Analysis (PCA), Type I ROS and localization of V-LNPs in the endoplasmic reticulum (ER) and mitochondria were identified as the most strongly associated with exposure of ICD markers. Type II ROS contributes the least to the exposure of ICD markers and in fact has a statistically significant inverse relationship with exposure of ICD markers HMGB1 (r=(−0.4421)-(−0.6079), p<0.0004) and HSP-70 (r=(−0.5912)-(−0.9212), p<0.0001). Conversely, Type I ROS contributes the most to the exposure of ICD markers with a statistically significant direct relationship with exposure of ICD markers HMGB1 (r=(0.7529)-(0.8252), p<0.0001) and HSP-70 (r=(0.6527)-(0.7987), p<0.0001). The role of calreticulin translocation was ambiguous; while there was a trend that resembled those of HMGB1 and HSP-70, the trend was not statistically significant. Calreticulin translocation, however, was associated only with lower PDT doses (IC₂₅ and IC₅₀) and lysosomal localization. The dependence on radical species for the exposure of ICD markers and the ambiguous involvement of calreticulin suggests that the oxidation targets (lipids, proteins, etc.) are important mediators of ICD induced by PDT using V-LNPs. Fine-tuning design features of novel V-LNPs, such as Type I ROS generation and predominantly ER and mitochondrial localization, provides a more robust induction of ICD.

### BRIEF DESCRIPTION OF DRAWINGS

FIG. 1A shows cryo-transmission electron microscopy images of preferred embodiments of lipid nanoparticles described herein, with a graphical representation of free and lipidated BPD formulated into solid lipid nanoparticles and a liposomal formulation.

FIG. 1B shows chemical structures of components used in preferred embodiments of lipid nanoparticles described herein.

FIG. 2 shows (A) a graphical representation of light-induced immunogenic cell death (ICD) using preferred embodiments of V-LNPs in pancreatic cancer cells, including subcellular localization, ROS production, and ICD induction and (B) a flow chart showing different conditions, attributes, and outputs used for Principal Component Analysis (PCA) for optimization of the lipid nanoparticles to maximum ICD efficiency.

FIG. 3 shows generation of reactive oxygen species by different LNP formulations and liposomal formulations measured using fluorescent probes, including (A and B) measuring singlet oxygen production using the fluorescent probe Singlet Oxygen Sensor Green (SOSG), λ_{Exc}=460 nm and λ_{Emi}=530 nm, and (C and D) measuring hydroxyl radical and peroxynitrite anion generation using fluorescent probe hydroxyphenyl fluorescein (HPF).

FIG. 4 shows quantification of Pearson's coefficient values for co-localized formulations of preferred embodiments of lipid nanoparticles in CT1BA5 cells within (A) Lysosomes, (B) Mitochondria, and (C) Endoplasmic reticulum (ER).

FIG. 5 shows exposure of the immunogenic cell death markers (A) HSP-70, (B) HMGB1 and (C) Calreticulin in CT1BA5 cells post PDT using 690 nm light with a fluence of 20 J/cm² and IC₅₀ BPD equivalent concentrations of a panel of preferred embodiments of lipid nanoparticles.

FIG. 6 shows representative scatter plots showing the relationships between the exposure of the immunogenic cell death marker (A,B) HSP-70, (C,D) HMGB-1, and (E,F) calreticulin at IC₅₀ BPD equivalent concentrations using different LNP and liposomal formulations and Type II ROS and Type I ROS.

FIG. 7 shows a heat map showing the Pearson's Coefficient matrix depicting the relationships between ROS generation (Type I and Type II), ICD markers (calreticulin, HSP-70, and HMGB1), and sub-cellular localization (lysosomes, mitochondria (Mito) and ER) for preferred embodiments of lipid nanoparticles.

FIG. 8 shows a graphical representation of the role of lipid nanoparticles according to preferred embodiments described herein in the exposure of ICD markers (HSP-70, HMGB1, and calreiculin) in CT1BA5 and 6620c1 cells and production of types of ROS.

FIG. 9 shows Principal Component Analysis (PCA) results, including (A) PC scores plot showing sample distribution along the principal components, highlighting clustering patterns and group separations, and (B) PCA loadings plot illustrating the contribution of individual variables to each principal component.

FIG. 10 shows a graphical representation of PDT-induced immunogenic cell death (HSP-70, calreticulin, and HMGB1 exposure) using Lipo 20:0 BPD-PC (SM-102) and Lipo 20:0 BPD-PC (without SM-102) in murine pancreatic cancer cells that produce varying levels of Type I and Type II ROS and exhibit varying degrees of subcellular localization and phototoxicity.

FIG. 11 shows representative cryo-Transmission Electron Microscopy (TEM) images of (A) Lipo 20:0 BPD-PC (with SM-102), (B) Lipo 20:0 BPD-PC (without SM-102).

FIG. 12 shows generation of reactive oxygen species (ROS) by light-activated liposomes containing BPD-PC measured using fluorescent probes, including (A) the ratio of HPF/SOSG, (B) the area under the curve of HPF/SOSG, (C and D) Singlet oxygen production, and (E and F) Hydroxyl radical and/or peroxynitrite anion generation.

FIG. 13 shows CT1BA5 cellular uptake of 20:0 BPD-PC delivered by Lipo 20:0 BPD-PC (without SM-102), Lipo 20:0 BPD-PC (SM-102), and LNP 20:0 BPD-PC (SM-102).

FIG. 14 shows Pearson's coefficient values for colocalization of Lipo 20:0 BPD-PC (with SM-102) and Lipo 20:0 BPD-PC (without SM-102) with markers for (A) Lysosomes, (B) Mitochondria, and (C) Endoplasmic reticulum (ER) in CT1BA5 cells.

FIG. 15 shows metabolic activity of CT1BA5 cells as determined by the Alamar Blue assay following (A) no PDT and (B) PDT using all the formulations activated by 690 nm light with a fluence of 20 J/cm².

FIG. 16 shows exposure of the ICD marker (A) HSP-70, (B) HMGB1 and (C) calreticulin in CT1BA5 cells post-PDT using 690 nm light with a fluence of 20 J/cm² and IC₂₅, IC₅₀, and IC₇₅ concentrations of Lipo 20:0 BPD-PC (SM-102) and Lipo 20:0 BPD-PC (without SM-102).

FIG. 17 shows (A) CT1BA5 tumor volumes of mice treated with light activated Lipo 20:0 BPD-PC (SM-102) and Lipo 20:0 BPD-PC (without SM-102) until day 20, and (B) Body weights of those mice demonstrating not systemic toxicity following treatment.

FIG. 18 shows (A) CT1BA5 tumor volumes of mice treated with light activated Lipo 20:0 BPD-PC (low ICD efficiency) or LNP BPD-cholesterol (high ICD efficiency) until day 40, (B) endpoint tumor volumes measured at the experimental endpoint, (C) Kaplan-Meier plots representing the probability of overall survival in mice, and (D) body weights.

FIG. 19A shows gene set enrichment analysis (GSEA) comparing tumors treated with exemplary lipid nanoparticles LNP BPD-Cholesterol PDT (high ICD efficiency) relative to Lipo 20:0 BPD-PC PDT (low ICD efficiency).

FIG. 19B shows immune cell-associated gene signatures enriched in exemplary lipid nanoparticles LNP BPD-Cholesterol PDT (high ICD efficiency) relative to Lipo 20:0 BPD-PC PDT (low ICD efficiency).

FIG. 20 shows (A) CT1BA5 tumor volumes of mice for control, α-PD-L1, LNP BPD-Cholesterol PDT, and LNP BPD-Cholesterol PDT+α-PD-L1 treatment groups, (B) tumors treated with the combination of LNP-mediated PDT and α-PD-L1, (C) Kaplan-Meier plots representing the probability of overall survival in mice, and (D) Body weights.

FIG. 21A shows gene set enrichment analysis (GSEA) comparing CT1BA5 tumors treated with LNP BPD-Cholesterol PDT+α-PD-L1 relative to α-PD-L1 alone.

FIG. 21B shows immune cell-associated gene signatures enriched in CT1BA5 tumors treated with LNP PDT+α-PD-L1 relative to α-PD-L1 alone.

### DETAILED DESCRIPTION OF PREFERRED EMBODIMENTS

The present disclosure relates to lipid nanoparticles for use in cancer therapy and methods for optimizing the features of lipid nanoparticles to enhance their tumor immune responses.

Visudyne®, a liposomal formulation of the hydrophobic photosensitizer BPD, was approved for wet age-related macular degeneration in the year 2000. Visudyne® is currently being investigated in numerous clinical trials for cancer treatment (NCT03033225, NCT02872064, NCT06381154). However, one of the major limitations of any lipid-based formulation of a benzoporphyrin derivative (BPD) is the leakage of photosensitizer into biological media. Anchoring a BPD to a lipid reduces the leakage of BPD from liposomes. Preferred embodiments disclosed herein include verteporfin-lipid nanoparticles (V-LNPs) or BPD-LNPs that may be liposomes or solid lipid nanoparticles, such as LNP 20:0 BPD-PC, LNP 16:0 BPD-PC, LNP Free BPD, LNP BPD-Cholesterol, and Lipo 20:0 BPD-PC containing unmodified verteporfin or verteporfin conjugated to cholesterol, 20:0 lyso PC, or 16:0 lyso PC. Their photophysical and photochemical properties, cellular uptake, metabolic activity, and their ability to induce ICD in murine pancreatic cancer cells (CT1BA5 and 6620c1) was evaluated.

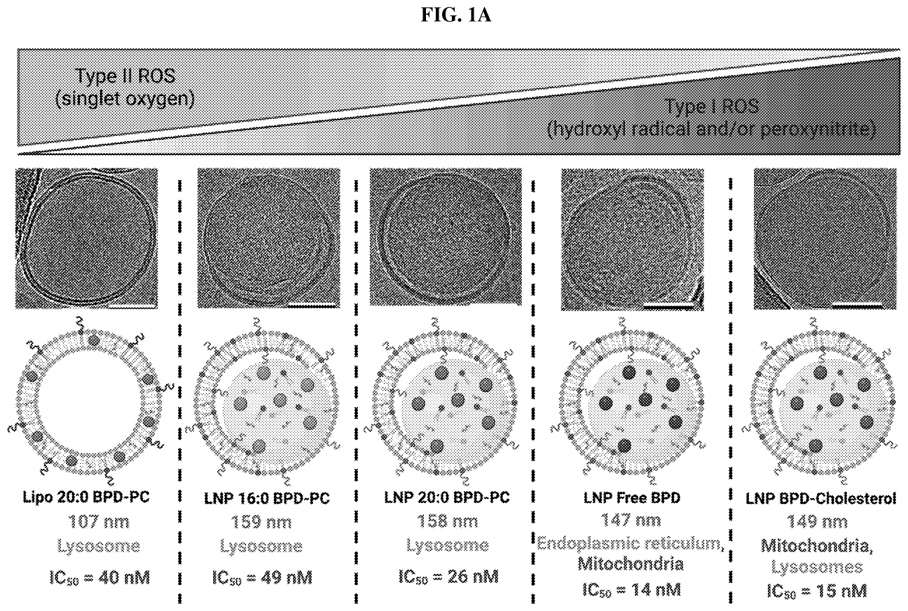

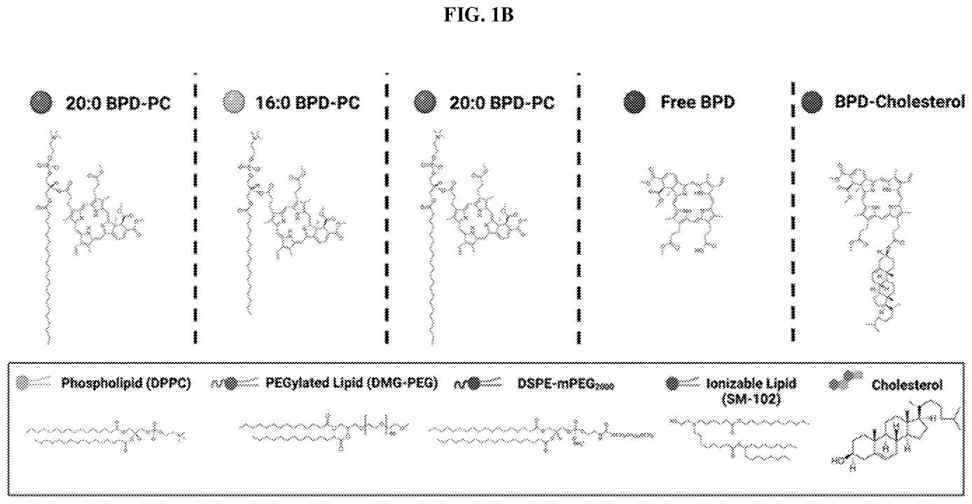

FIG. 1A shows cryo-transmission electron microscopy images of preferred embodiments of V-LNPs; scale bar, 50 nm. along with a graphical representation of free and lipidated BPD formulated into solid lipid nanoparticles and a liposomal formulation. FIG. 1B shows chemical structures of 20:0 BPD-PC, 16:0 BPD-PC, Free BPD, BPD-Cholesterol and the lipids DPPC, DMG-PEG, DSPE-mPEG₂₀₀₀, SM-102, and cholesterol used in the formulations.

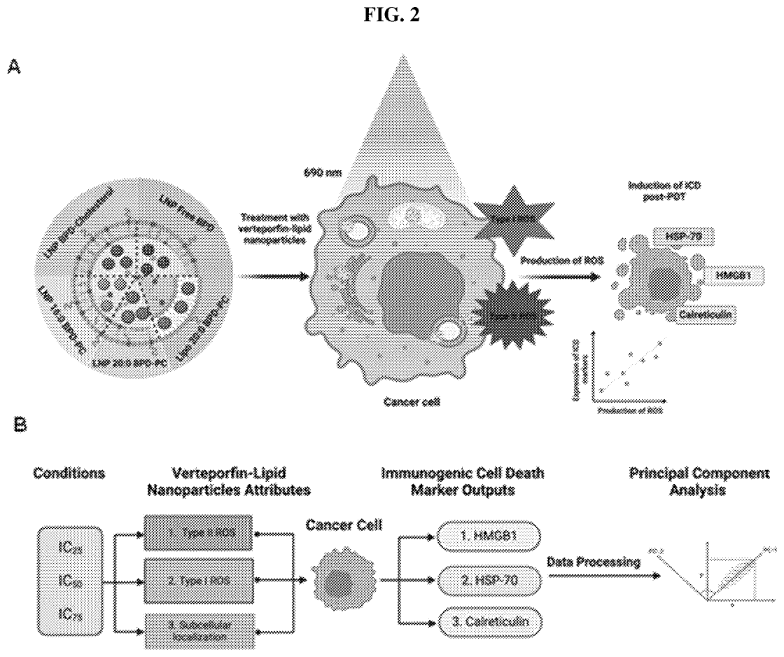

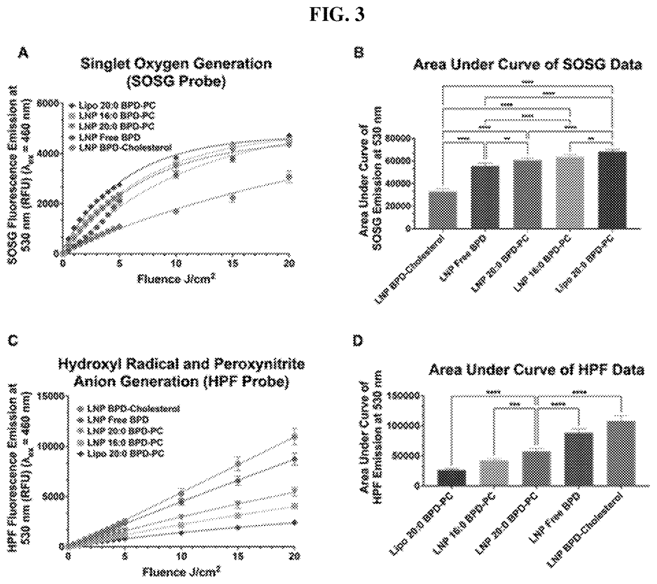

FIG. 2 shows (A) a graphical representation of PDT using preferred embodiments of V-LNPs in pancreatic cancer cells, including subcellular localization, ROS production, and ICD induction and (B) a flow chart showing different conditions, attributes, and outputs used for Principal Component Analysis (PCA) for optimization of the lipid nanoparticles. As shown in FIG. 2, the efficiency of all the V-LNPs to induce ICD using three different BPD equivalent concentrations (IC₂₅, IC₅₀, and IC₇₅) post-irradiation with a 690 nm wavelength light was assessed. An unsupervised machine learning algorithm, Principal Component Analysis (PCA), was used to elucidate the relationships between subcellular localization, the type of ROS generation, and the induction of ICD markers. The PCA data highlighted that Type I ROS and localization of V-LNPs in the endoplasmic reticulum (ER) and mitochondria are strongly associated with the upregulation of HSP-70 and HMGB1. Fine-tuning the type of photochemical ROS generated and their subcellular localization using a panel of novel V-LNPs allows for the optimization of V-LNPs with enhanced tumor immune responses through higher exposure of ICD markers using PDT.

In preferred embodiments, the use of ionizable lipids is preferred to enhance Type I photochemistry, lower Type II photochemistry, and increase mitochondrial localization of the PS, which is conducive to light-activated ICD. In further preferred embodiments, the ionizable lipids may be the clinical ionizable lipids SM-102, DLin-MC3-DMA, or ALC-0315, among other ionizable lipids in experimental or clinical trial use, such as C12-200, KC2, L319, LP01, A18-Iso5-2DC18, OF-02 or OF-03, or combinations thereof.

In additional preferred embodiments, solid lipid nanoparticle configuration is preferred over liposome configuration for enhanced Type I photochemistry and lower Type II photochemistry. These are conducive to light-activated ICD. Preferred embodiments described herein are not limited to the use of lipid nanoparticles. The photoactivatable nanoparticles can be organic or inorganic nanoparticles, self-assembled nanoparticles, crystalline nanoparticles, amorphous nanoparticles, lipid micelles, polymer micelles, hybrid micelles, polymeric nanoparticles, protein-based nanoparticles, hybrids of lipid nanoparticles and polymeric nanoparticles, metal-organic framework nanoparticles, matrix type nanoparticles, as well as nanodroplets, nanoemulsions, and other suitable nanoparticles, or combinations thereof.

Accordingly, preferred embodiments described herein include photoactivatable nanoparticles for use as therapeutic agents in immunotherapy treatments, comprising lipid nanoparticles, wherein the lipid nanoparticles are liposomes or solid lipid nanoparticles, and wherein the lipid nanoparticles comprise phospholipids and lipids bound to polyethylene glycol. The lipid nanoparticles also comprise photosensitizers incorporated within the nanoparticles, wherein the photosensitizers are benzoporphyrin derivative or any suitable chemical variant of benzoporphyrin derivative, and wherein the photoactivatable nanoparticles generate reactive oxygen species through light activation of the photosensitizer. In additional preferred embodiments the benzoporphyrin derivative may be unbound or it may be bound to phosphatidylcholine or cholesterol. Chemical variants of benzoporphyrin derivative may include structural isomers and regioisomers, as well as analogs and derivatives thereof.

In additional preferred embodiments, the benzoporphyrin derivative of the lipid nanoparticles may be bound to 20:0 lysophosphatidylcholine or 16:0 lysophosphatidylcholine. In further preferred embodiments, the lipid nanoparticles further comprise ionizable lipids, such as SM-102. The photoactivatable nanoparticles preferably generate Type I reactive oxygen species (ROS) and preferably localize to the endoplasmic reticulum (ER) and mitochondria of the cancer cells. The photoactivatable nanoparticles also induce immunogenic cell death.

Additional preferred embodiments of the present disclosure include a method for immunotherapy in a subject. The method preferably comprises delivering preferred embodiments of the photoactivatable nanoparticles described herein to cancer cells of the subject for use as therapeutic agents, wherein the photoactivatable nanoparticles comprise lipid nanoparticles and photosensitizers incorporated within the lipid nanoparticles, wherein the lipid nanoparticles are liposomes or solid lipid nanoparticles, wherein the lipid nanoparticles comprise phospholipids and lipids bound to polyethylene glycol, wherein the photosensitizers are benzoporphyrin derivative. In a further step, the photosensitizers are activated though light activation, whereby the photoactivatable nanoparticles generate reactive oxygen species and induce immunogenic cell death in the cancer cells. The photoactivatable nanoparticles may also trigger release of immunogenic cell death markers in the cancer cells, such as calreticulin, HMGB1, and HSP-70.

In additional preferred embodiments of the method for immunotherapy in a subject, an additional immune checkpoint inhibitor may be delivered to the subject with the photoactivatable nanoparticles. Preferred examples of additional immune checkpoint inhibitors include clinical antibodies such as anti-PD-1, anti-PD-L1, anti-CTLA-4, anti-LAG-3, as well as emerging antibodies such as anti-TIGIT, anti-TIM-3, anti-VISTA (PD-1H/B7-H5), anti-B7-H3, anti-B7-H4, anti-HHLA2 (B7-H7), anti-BTLA, anti-CD96, anti-CD160, anti-CD39, anti-CD73, anti-CD47/SIRPα, and combinations thereof. Checkpoint inhibitors anti-PD-1 and anti-PD-L1 are part of the same immune checkpoint pathway, in that they both block the PD-1/PD-L1 pathway by inhibiting the binding of PD-1 to PD-L1. In additional preferred embodiments, the additional immune checkpoint inhibitor is conjugated to or encapsulated within the lipid nanoparticles.

Additional preferred embodiments disclosed herein include a method for designing photoactivatable nanoparticles having enhanced activity as therapeutic agents. The method preferably includes a step of using a data-driven statistical tool or unsupervised machine learning algorithm to evaluate properties and attributes of photoactivatable nanoparticles and produce evaluation results, wherein the properties and attributes comprise photophysical and photochemical properties. The properties and attributes of the photoactivatable nanoparticles preferably include the properties and attributes of all preferred embodiments of the photoactivatable nanoparticles described herein. The properties and attributes of the photoactivatable nanoparticles may comprise one or more of the inclusion of liposome lipid nanoparticles, solid lipid nanoparticles, unbound benzoporphyrin derivative, benzoporphyrin derivative bound to 20:0 lysophosphatidylcholine, benzoporphyrin derivative bound to 16:0 lysophosphatidylcholine, benzoporphyrin derivative bound to cholesterol, and lipid nanoparticles comprising ionizable lipids, as well as administration of the photoactivable nanoparticles with anti-PD-L1 antibody. In a preferred embodiment, the data-driven statistical tool or unsupervised machine learning algorithm is Principal Component Analysis.

In a further step, the evaluation results of the data-driven statistical tool or unsupervised machine learning algorithm are used to predict relationships between the properties and attributes of the photoactivatable nanoparticles and efficiency properties of the various photoactivatable nanoparticles. The efficiency properties are preferably based on suitable tests and analyses performed to evaluate the effectiveness of the various properties and attributes of photoactivatable nanoparticles, such as those described herein. In preferred embodiments the efficiency properties comprise subcellular localization, such as subcellular localization to the endoplasmic reticulum (ER) and mitochondria of the cancer cells, the type of ROS generation, such as Type I ROS generation, and induction of immunogenic cell death markers, such as calreticulin, HMGB1, and HSP-70. A further preferred step includes designing photoactivable nanoparticles having optimized efficiency properties and enhanced activity as therapeutic agents.

Additional preferred embodiments are directed to a method for enhancing immune cell infiltration and reducing immunosuppression in cancer tissue in a subject by delivering preferred embodiments of the photoactivatable nanoparticles described herein and activating the photosensitizers of the photoactivatable nanoparticles, whereby the photoactivatable nanoparticles generate reactive oxygen species and enhance immune cell infiltration and reduce immunosuppression in the subject. In additional preferred embodiments, a further step includes delivering immune cells to the cancer tissue of the subject in conjunction with the photoactivatable nanoparticles. Preferred examples of the immune cells include macrophage or natural killer (NK) cells, as well as immune cells derived from adoptive cell therapy or chimeric antigen receptor cell therapy.

Additional discussion of exemplary preferred embodiments is included in the examples below. U.S. Patent Application Publication No. US 2025/0205358 is incorporated by reference in its entirety herein.

### Example 1

### Material and Methods

Synthesis of 20:0 BPD-PC, 16:0 BPD-PC, and BPD-Cholesterol: BPD was anchored to the 1-arachidoyl-2-hydroxy-sn-glycero-3-phosphatidylcholine (20:0 lyso PC), (1-palmitoyl-2-hydroxy-sn-glycero-3-phosphatidylcholine (16:0 lyso PC) and cholesterol through Steglich esterification. Briefly, BPD (U.S. Pharmacopenia), 4-(Dimethylamino) pyridine (DMAP, Sigma-Aldrich), 1-ethyl-3-(3-dimethylaminopropyl) carbodiimide (EDC; Sigma-Aldrich), N,N-Diisopropylethylamine (DIPEA, Sigma-Aldrich), and 16:0 lyso PC were mixed at molar ratios of 1:5:50:25:60, respectively, in 5 mL of dichloromethane (DCM; Fischer Scientific, high-performance liquid chromatography [HPLC] grade) and stirred at 2500 RPM on a magnetic stirrer for 72 h at room temperature to synthesize 16:0 BPD-PC. The 16:0 BPD-PC was purified using preparatory thin-layer chromatography and extracted in a 2:1 dichloromethane/methanol mixture. The extracted 16:0 BPD-PC was then filtered using a 0.22 μm polytetrafluoroethylene (PTFE) filter and stored in chloroform at −20° C. in the dark. Using the same procedure and reaction stoichiometry, BPD was also conjugated to 20:0 lyso PC and cholesterol.

Lipo 20:0 BPD-PC was synthesized. Briefly the lipids, 2-Dipalmitoyl-sn-glycero-3-phosphatidylcholine (DPPC, Avanti), cholesterol, 1,2-Distearoyl-sn-Glycero-3-Phosphoethanolamine with conjugated methoxyl poly(ethylene glycol) (DSPE-mPEG₂₀₀₀, Avanti), and 20:0 BPD-PC were mixed in chloroform at a molar ratio of 0.675:0.300:0.015:0.010, respectively. Lipo 20:0 BPD-PC was prepared by a conventional thin-film hydration method by hydrating the thin-film of lipids with Dulbecco's Phosphate Buffered Saline (DPBS; no Calcium, no Magnesium; Corning) followed by ultrasonication (Ultrasonic probe sonicator; Fisher Scientific) for a total of 30 min (20 s on/40 s off cycles) at 42° C. in the dark.

LNP 16:0 BPD-PC was prepared. Briefly, all the lipid components, including 1,2-Dipalmitoyl-sn-glycero-3-phosphatidylcholine (DPPC), 1,2-Dimyristoyl-sn-glycero-3-phosphatidylcholine (DMG-PEG), cholesterol, the ionizable lipid SM-102 9-heptadecanyl 8-{(2-hydroxyethyl)[6-oxo-6-(undecyloxy)hexyl]amino}octanoate, and the 16:0 BPD-PC were mixed at a molar ratio of 0.090:0.015:0.385:0.500:0.010, respectively. Further, using a syringe pump, the lipid solution was added dropwise using a flow rate of 0.8 mL/min into 0.75 mL of citrate buffer (pH 4.0). After the dropwise addition, the LNP 16:0 BPD-PC solution was stirred on a magnetic stir plate at 2500 RPM for 18 h. LNP 16:0 BPD-PC was then dialyzed using a 100 kDa dialysis tube by replacing the buffer solution with 1×DPBS (pH 7.4; no Calcium, no Magnesium; Corning) and stirred at 150 RPM. During the 24 h dialysis process, DPBS solution was replaced after every 8 h. The resulting LNP 16:0 BPD-PC solution was stored at 4° C. in the dark. LNP 20:0 BPD-PC, LNP BPD-Cholesterol and LNP Free BPD were also prepared with the same protocol, with 16:0 BPD-PC replaced by 20:0 BPD-PC, BPD-Cholesterol, and Free BPD, respectively.

Optical characterization: The hydrodynamic size and zeta potential of LNP 20:0 BPD-PC, LNP 16:0 BPD-PC, LNP BPD-cholesterol, LNP Free BPD, and Lipo 20:0 BPD-PC were measured using dynamic light scattering (DLS). UV-Vis spectrophotometry was used to measure the BPD equivalent concentration in the V-LNPs using ε₆₈₇ nm=34,895 M⁻¹ cm⁻¹ in dimethyl sulfoxide (DMSO, Sigma-Aldrich). For the absorbance spectra, 500 μL of a 5 μM BPD equivalent concentration of all the V-LNPs (LNP 20:0 BPD-PC, LNP 16:0 BPD-PC, LNP BPD-Cholesterol, LNP Free BPD, and Lipo 20:0 BPD-PC) was measured in DPBS using UV-Vis spectrophotometry. For fluorescence emission measurements, a 5 μM BPD equivalent concentration of LNP 16:0 BPD-PC, LNP BPD-Cholesterol, and LNP Free BPD was prepared in DPBS and serum (Fetal Bovine Serum, S11150; R&D Systems). 100 μL of the sample was placed in triplicates into a transparent bottom white well 96-plate (Corning). The fluorescence emission of each sample was then measured using a Tecan Spark plate reader with an excitation wavelength of 435 nm and emission wavelengths ranging from 650 nm to 750 nm in 2 nm increments. The measurement settings included a manual gain of 100, 0 μs lag time, an integration time of 40 μs, and 25 flashes per read.

Cryo-Electron microscopy: 3-4 μL of the V-LNPs solution were added to Lacey carbon grids (300-mesh; Ted Pella, Inc.) that were negatively glow-discharged for 80 s at 30 mA. Excess sample was removed by blotting once for 4 s with filter paper (Ted Pella, Inc.). Then, the grid was plunge-frozen in liquid ethane cooled by liquid nitrogen using a Leica GP2 automatic plunge freezer (Leica Microsystems). The vitrified samples were imaged using a Talos Arctica or a Glacios 200 kV cryo-transmission electron microscope (cryo-TEM) (Thermo Fisher Scientific) equipped with a K3 camera (Gatan). Serial EM software v4.1 or higher was used to collect images under low-dose conditions with a pixel size of ~1 Å/pixel. A total of 50 frames were recorded for each image over a 2.5 s exposure time with an average dose rate of ~20 e-/pixel/s.

Physical stability and photostability measurements: The physical stability of LNP 16:0 BPD-PC, LNP BPD-Cholesterol, and LNP Free BPD was assessed under three different conditions: at 4° C. in DPBS and at 37° C. in both DPBS and serum-containing Dulbecco's Modified Eagle Medium (DMEM) for 7 days. 10 μM BPD equivalent concentrations of LNP 16:0 BPD-PC, LNP BPD-Cholesterol and LNP Free BPD were incubated at 4° C. in DPBS and at 37° C. in DPBS and 10% serum containing DMEM media. Following incubation, the hydrodynamic sizes and polydispersity indices (PDIs) were monitored daily to assess any changes in size and PDI over the course of 7 days using DLS.

5 μM BPD equivalent concentrations of LNP 20:0 BPD-PC, LNP 16:0 BPD-PC, LNP BPD-Cholesterol, LNP Free BPD and Lipo 20:0 BPD-PC was prepared in DPBS or serum to evaluate photostability. 100 μL of sample was placed in a clear-bottom white walled 96-well plate (Corning). The fluorescence emission of BPD was measured after irradiation with 690 nm light (Biolambda) administered to the top of the plates in incremental doses of 0 J/cm², 0.5 J/cm², 1 J/cm², 1.5 J/cm², 2 J/cm², 2.5 J/cm², 3 J/cm², 3.5 J/cm², 4 J/cm², 4.5 J/cm², 5 J/cm², 10 J/cm², 15 J/cm², and 20 J/cm² at an irradiance of 17.86 mW/cm².

ROS generation: The generation of singlet oxygen was measured using the fluorometric probe Singlet Oxygen Sensor Green (SOSG, Fisher Scientific). Hydroxyl radicals and peroxynitrite anions were measured using the fluorometric probe hydroxyphenyl fluorescein (HPF, Fisher Scientific). For the SOSG assays, 10 μL of 50 μM SOSG was added to 100 μL each of a 5 μM BPD equivalent concentration of LNP 20:0 BPD-PC, LNP 16:0 BPD-PC, LNP BPD-Cholesterol, LNP Free BPD and Lipo 20:0 BPD-PC prepared in DPBS. The fluorescence emission was then measured at 530 nm using an excitation wavelength of 460 nm with a Tecan Spark plate reader after irradiation with 690 nm light with increments of 0.5 J/cm², to a total fluence of 20 J/cm², at an irradiance of 17.86 mW/cm². To measure hydroxyl radicals and peroxynitrite anions, 20 μL of 200 μM HPF was added to 100 μL each of a 5 μM BPD equivalent concentration of LNP 16:0 BPD-PC, LNP 20:0 BPD-PC, LNP BPD-Cholesterol, LNP Free BPD, and Lipo 20:0 BPD-PC prepared in DPBS. The fluorescence emission was then measured at 530 nm using an excitation wavelength of 460 nm with a Tecan Spark plate reader after irradiation with 690 nm light with increments of 0.5 J/cm² to a total fluence of 20 J/cm² at an irradiance of 17.86 mW/cm².

Cell culture: CT1BA5 and 6620c1 cells used in this study were a kind gift from the Brekken Lab at the University of Texas Southwestern Medical Center (UTSW). CT1BA5 cells are an isogenic pancreatic cancer cell line derived from KPf C (Kras^{LSL-G12D}; Trp53^{f l/f l}; PDX^{Cre/+}) mice. KPC cells 6620c1 on C57BL/6 mice were developed at Dr. Ben Stanger's lab (University of Pennsylvania). Cells were cultured in DMEM media supplemented with 10% fetal bovine serum (FBS) and 1× penicillin/streptomycin.

Measuring the light induced phototoxicity: CT1BA5 and 6620c1 cells (1,500 cells per well) were seeded in clear bottom transparent 96-well plate (Corning) and incubated for 24 h at 37° C. After 24 h, cells were incubated with media containing BPD equivalent concentrations of LNP 20:0 BPD-PC, LNP 16:0 BPD-PC, LNP BPD-Cholesterol, LNP Free BPD, and Lipo 20:0 BPD-PC ranging from 0.100 nM to 10,000 nM. Following 24 h incubation, cells were irradiated using 690 nm light at a fluence of 20 J/cm² and an irradiance of 17.86 mW/J/cm². After 48 h incubation, media content from each well was removed and replaced with 100 μL of 0.3 mg/mL of 3-(4,5-dimethylthiazol-2-yl)-2,5-diphenyltetrazolium bromide (MTT dye, Sigma-Aldrich). Further, cells were incubated for at least 90 min or until visible formazan crystals formed. All the contents of each well were then removed and 100 μL of DMSO was added to each well to dissolve the formazan crystals. Using a Tecan Spark plate reader, absorbance was then measured at a wavelength of 555 nm.

Cellular uptake: 50,000 CT1BA5 cells were seeded in a transparent bottom white walled 96-well plate (Corning) and incubated at 37° C. After 24 h, cells were then incubated with a 250 nM of BPD equivalent concentration of LNP 20:0 BPD-PC, LNP 16:0 BPD-PC, LNP BPD-Cholesterol, LNP Free BPD, and Lipo 20:0 BPD-PC. After 24 h of incubation, cells were washed three times with 100 μL of DPBS. Further, 100 μL of DPBS containing 1% Triton X-100 was added to each well. The plate was covered with aluminum foil and placed on a shaker for 1 h. The fluorescence emission of each well was measured using a Tecan Spark plate reader with an excitation wavelength of 435 nm and an emission wavelength of 698 nm, a gain of 150, and an integration time of 100 μs. The BPD equivalent concentration of each sample was extracted from a standard curve prepared for each formulation. The standard curve for LNP 20:0 BPD-PC, LNP 16:0 BPD-PC, LNP BPD-Cholesterol, LNP Free BPD, and Lipo 20:0 BPD-PC were generated by making serial range dilutions (with the highest concentration of 250 nM and lowest concentration of 0.25 nM) of BPD equivalent concentrations of each formulation in DPBS containing 1% Triton X-100.

Determining subcellular localization: 50,000 CT1BA5 cells were seeded in a 96-well transparent glass bottom black walled plate (Celvis) and incubated at 37° C. for 24 h. After 24 h, cells were incubated with media containing a 2,000 nM BPD equivalent concentration of LNP 20:0 BPD-PC, LNP 16:0 BPD-PC, LNP BPD-Cholesterol, LNP Free BPD, and Lipo 20:0 BPD-PC. After 24 h incubation, the media content in each well was replaced with fresh media containing either 1 μg/ml of Hoechst (nuclei tracker; Cell Signaling Technology), 50 nM of the Lyso-tracker (lysosome tracker; Cell Signaling Technology), 50 nM of the Mito-tracker (mitochondria tracker; Cell Signaling Technology), or 1 μM of the ER-tracker (endoplasmic reticulum tracker; Cell Signaling Technology). Cells were further incubated for 1 h in the dark at 37° C. Thereafter, cells were further washed three times using fresh media before imaging for colocalization. An Olympus FV300RS Confocal Laser Scanning Microscope was used to determine colocalization at a 100× oil immersion objective with a 405 nm laser for Hoechst excitation, a 488 nm laser for Lysotracker excitation, a 568 nm laser for Mitotracker or ER tracker excitation, and a 647 nm laser for BPD excitation in all the V-LNPs.

Measuring immunogenic cell death: To assess ICD, CT1BA5 and 6620c1 cells were trypsinized and seeded in a 6-well plate at a density 45,000 cells per well and then incubated at 37° C. After 24 h incubation, the media in each well was replaced with fresh media containing IC₂₅, IC₅₀, and IC₇₅ of BPD equivalent concentrations of LNP 20:0 BPD-PC, LNP 16:0 BPD-PC, LNP BPD-Cholesterol, LNP Free BPD, and Lipo 20:0 BPD-PC. For the 6620c1 cell line, cells were tested only with IC₅₀ BPD equivalent concentration of all the V-LNPs. Following 24 h incubation, the cells were irradiated with 690 nm light from the top of the plate with a fluence of 20 J/cm² and an irradiance of 17.86 mW/cm². After another incubation of 24 h, cells were trypsinized, transferred to Eppendorf tubes, and washed with 1× cold DPBS. To reduce non-specific binding of antibody, cells were stained with 50 μL of TruStain (0.5 mg/mL, 1:5 dilution in phosphate azide buffer (PAB), BioLegend; 101320) and placed in ice for 15 mins in the dark. After incubation, to measure calreticulin and HSP-70 exposure, cells were resuspended in 50 μL of anti-calreticulin antibody (0.406 μg/mL, 1:400 dilution in antibody dilution buffer, Cell Signaling Technology; D3E6) or 50 μL of anti-HSP-70 antibody (20 μg/mL, 1:400 dilution in antibody dilution buffer, Cell Signaling Technology; 4872S) and incubated for 15 min in the dark on ice. After incubation, cells were washed with PAB and resuspended in 50 μL of secondary antibody conjugated with AF594 (2 mg/mL, 1:500 dilution in PAB, Cell Signaling Technology; 8889S). Following 15 min of incubation on ice in dark, cells were washed with PAB and then fixed with 2% formalin at room temperature. The cells were washed again with PAB and resuspended in 300 μL of PAB for flow cytometry analysis. To determine the median AF594 fluorescence emission corresponding to calreticulin and HSP-70 levels, a 561 nm laser and a 610/20 nm bandpass detector was used.

To measure HMGB1 release, a Lumit™ HMGB1 immunoassay kit (Promega) was used. CT1BA5 and 6620c1 cells were trypsinized and seeded in a 96-well plate at a density of 20,000 cells per well. The cells were incubated at 37° C. for 24 h. Following incubation, the media in each well was replaced with fresh media containing IC₂₅, IC₅₀, and IC₇₅ concentrations of the BPD equivalent for LNP 20:0 BPD-PC, LNP 16:0 BPD-PC, LNP BPD-Cholesterol, LNP Free BPD, and Lipo 20:0 BPD-PC. For the 6620c1 cell line, cells were tested only with IC₅₀ BPD equivalent concentration of all the V-LNPs. After an additional 24 h incubation, the cells were irradiated with 690 nm light from the top of the plate at a fluence of 20 J/cm² and an irradiance of 17.86 mW/cm². Further, 20 L of 5×HMGB1 antibody solution (containing Anti-hHMGB1 mAV-SmBIT and Anti-hHMGB1 mAB-LgBiT) was added to each well and incubated for 60-90 min. Following antibody incubation, 25 μL of a 1:20 dilution of Lumit™ detection substrate was added to each well, and the plate was mixed using a shaker at 300-500 RPM. The plate was then incubated for 3-5 min, and luminescence readings were recorded using a multi-plate reader.

Principle Component Analysis (PCA): PCA was performed to reduce data dimensionality and identify relationships among different V-LNPs based on their ROS generation, subcellular localization, and their ability to induce exposure of ICD marker. The analysis was conducted using GraphPad Prism (GraphPad Software, Boston, MA). To determine the contribution of each principal component (PC) to the total variance, eigenvalues were computed. PCA scores were calculated to position each formulation within the PCA space, enabling clustering analysis based on shared characteristics. A loadings plot was used to assess the contribution of individual variables to each PC. Additionally, a Pearson correlation matrix was computed to evaluate associations between variables and a heatmap was generated to visualize these correlations.

### Results and Discussion

Synthesis and characterization: The hydrodynamic sizes, PDIs, and ζ-potentials of LNP 20:0 BPD-PC, LNP 16:0 BPD-PC, LNP BPD-Cholesterol, LNP Free BPD, and Lipo 20:0 BPD-PC are summarized below in Table 1. The hydrodynamic sizes and PDIs of LNP 16:0 BPD-PC, LNP Free BPD, and LNP BPD-Cholesterol were 159 nm and 0.1, 147 nm and 0.1, and 149 nm and 0.09, respectively. These values fall within the size range of 100-180 nm which is commonly reported in the literature for the size of LNPs. PDIs were all below 0.2 which indicates that the formulations were relatively monodispersed. As shown in Table 1, the ζ-potentials of LNP 16:0 BPD-PC, LNP Free BPD, and LNP BPD-Cholesterol were −1.13 mV, −1.89 mV, and −1.37 mV, respectively. These values are near neutral because SM-102, a cationic lipid, remains uncharged in neutral saline solution. The hydrodynamic sizes, PDIs, and ζ-potentials for LNP 20:0 BPD-PC and Lipo BPD-PC were adapted from literature.

TABLE 1
Summary of the physical characterization of different V-LNPs.

| | Hydrodynamic | Polydispersity | |
|---|---|---|---|
| Formulation of | Diameter | Index | ζ-Potential |
| Photosensitizer | (nm ± S.D.) | (P.D.I. ± S.D.) | (mV ± S.D.) |
| Lipo 20:0 BPD-PC | 107 ± 1.0 | 0.13 ± 0.01 | −1.56 ± 0.13 |
| LNP Free BPD | 147 ± 1.0 | 0.10 ± 0.01 | −1.89 ± 0.18 |
| LNP BPD-Cholesterol | 149 ± 0.9 | 0.09 ± 0.01 | −1.37 ± 0.12 |
| LNP 20:0 BPD-PC | 158 ± 1.0 | 0.09 ± 0.02 | −4.30 ± 0.18 |
| LNP 16:0 BPD-PC | 159 ± 0.5 | 0.10 ± 0.01 | −1.13 ± 0.17 |

In Table 1 above, values are mean, n=3 (±SD). Cryo-transmission electron microscopy (cryo-TEM) was used to visualize the structural morphology of the V-LNPs. Previous studies have shown that in LNPs, PEG-lipids, DSPC, and cholesterol typically partition to the outer monolayer, while ionizable lipids accumulate in the LNP interior. Similarly, the micrographs revealed that LNPs containing 20:0 BPD-PC, 16:0 BPD-PC, and BPD-cholesterol exhibited a well-defined multilamellar structure, consisting of amorphous lipid aggregates coated with partially fused mono-, bi-, and multi-lipid layers. In contrast, the liposomal formulation of 20:0 BPD-PC displayed well-organized bilayers, consistent with the expected structural characteristics of liposomes.

The raw absorbance spectra of LNP 16:0 BPD-PC, LNP Free BPD, and LNP BPD-Cholesterol at a 5 μM BPD equivalent concentration in DPBS was studied. Compared to Lipo BPD-PC, all the LNP formulations exhibited light scattering. It is well known that large sized particles scatter more light. Here, the difference in scattering can be attributed to the larger size of LNP compared to liposomes. To account for the effects of scattering, the molar extinction coefficient was back-calculated using the Beer-Lambert Law for LNP 16:0 BPD-PC, LNP Free BPD, and LNP BPD-Cholesterol. Previously, the molar extinction coefficient of LNP 20:0 BPD-PC in DPBS was calculated as ε₆₈₇ nm=24,744 M⁻¹ cm⁻¹, while Lipo 20:0 BPD-PC had a molar extinction coefficient of ε₆₈₇ nm=21,032 M⁻¹ cm^{-1.34} Here, the molar extinction coefficients of LNP 16:0 BPD-PC (ε₆₈₇ nm=27,900 M⁻¹ cm⁻¹), LNP Free BPD (ε₆₈₇ nm=34,460 M⁻¹ cm⁻¹), and LNP BPD-Cholesterol (ε₆₈₇ nm=30,268 M⁻¹ cm⁻¹) were all measured in DPBS. BPD has an extinction coefficient of ε₆₈₇ nm=34,895 M⁻¹ cm⁻¹ in DMSO as it dissolves completely in DMSO. The lower molar extinction coefficient in DPBS suggests a dampening effect on the Q-bands of BPD in all the V-LNPs. This can occur due to the formation of J-aggregates of BPD in the formulations. Further, fluorescence emission of all the V-LNPs differed between the formulations in DPBS and in serum. LNP Free BPD exhibited the highest fluorescence, while Lipo 20:0 BPD-PC exhibited the lowest fluorescence emission in both DPBS and serum. This observation aligns with the literature, elucidating that face-to-face stacking and static quenching in liposomal bilayers, compared to LNP formulations, lead to lower fluorescence signals for porphyrins like BPD.

As a photosensitizer is excited by light during PDT, it generates ROS. These reactive oxygen radicals can then further react with the photosensitizer itself and photochemically degrade it. As such, the degraded photoproducts of the photosensitizer do not have the same fluorescence properties and PDT efficacy. The photostability of LNP 16:0 BPD-PC, LNP Free BPD, and LNP BPD-Cholesterol in DPBS and serum after irradiation with 690 nm light were evaluated and compared with previously published data of LNP 20:0 BPD-PC and Lipo 20:0 BPC-PC. LNP 20:0 BPD-PC was the most photostable, while LNP Free BPD was the least photostable in DPBS compared to all the formulations. This finding is consistent with the literature, which shows that free BPD easily leaks out of the liposomal formulation and reduces its photostability compared to the liposomal formulation of 20:0 BPD-PC. A different trend was observed in serum, where LNP 20:0 BPD-PC was the least photostable and Lipo 20:0 BPD-PC was the most photostable among all the formulations. This relationship likely arises because 20:0 BPD-PC leaches more readily from liposomes into the serum, where serum components help shield 20:0 BPD-PC from photobleaching by rapidly scavenging the generated ROS.

The stability of the V-LNPs during storage and in serum containing media was also studied. To assess the changes in hydrodynamic sizes and PDIs under different conditions, LNP 16:0 BPD-PC, LNP BPD-Cholesterol, and LNP Free BPD were incubated in DPBS at 4° C. and 37° C., as well as in serum at 37° C. All the V-LNPs remained relatively stable in both DPBS and serum containing media. The hydrodynamic diameters and PDIs of LNP Free BPD, LNP 16:0 BPD-PC, and LNP BPD-Cholesterol increased only marginally over 7 days under all conditions. This demonstrates that V-LNPs were stable in DPBS and serum containing media.

Quantifying the light-induced production of Type I and Type II reactive oxygen species by the verteporfin-lipid nanoparticles: Photosensitizers depend on Type I or Type II photochemistry for PDT effects. Type I reactions generate oxygen radicals, such as hydroxyl radicals, peroxynitrite anions, and superoxide anions, via electron transfer between photosensitizers and substrates, while Type II reactions produce singlet oxygen through energy transfer by excited photosensitizers to surrounding oxygen molecules. Most photosensitizers, including BPD, are Type II dominant, which is based on generating singlet oxygen using surrounding oxygen. Recently, studies have demonstrated that photosensitizers generating Type I ROS can enhance antitumor immune responses by effectively triggering ICD. In another recent study, supramolecular photosensitizer BSA@TPE-BTSCP nanoparticles was shown to enhance Type I ROS production. These Type I ROS producing nanoparticles can effectively induce various programmed cell death pathways, resulting in the induction of ICD by the release of DAMPs such as HMGB1, HSP-70, and ATP. Their study also confirmed increased T-cell activation and infiltration in 4T1 murine breast cancer cells. A phenothiazine-based Type I photosensitizer (Rh-PTZ) has also been designed for photoimmunotherapy. Rh-PTZ produced large amounts of Type I ROS and targeted mitochondria, inducing ICD via the release of calreticulin, HMGB1, and ATP in 4T1 murine breast cancer cells.

The relationship between the type of ROS produced by embodiments of V-LNPs and ICD was studied. FIG. 3 shows generation of reactive oxygen species by different LNP formulations and liposomal formulations measured using fluorescent probes, including (A and B) measuring singlet oxygen production using the fluorescent probe Singlet Oxygen Sensor Green (SOSG), λ_{Exc}=460 nm and λ_{Emi}=530 nm, and (C and D) measuring hydroxyl radical and peroxynitrite anion generation using fluorescent probe hydroxyphenyl fluorescein (HPF), λ_{Exc}=460 nm and λ_{Emi}=530 nm. All data are presented as mean±S.D., statistical significance was calculated using a one-way ANOVA test on GraphPad Prism v10.4.1, *: P<0.1, **: P<0.01, ***: P<0.001, ****: P<0.0001.

As for ROS production, singlet oxygen generation was measured using the SOSG probe, while hydroxyl radical and peroxynitrite anion generation was measured using the HPF probe. SOSG and HPF are fluorometric probes that demonstrate an increase in fluorescence signals when reacted with singlet oxygen and hydroxyl radicals and peroxynitrite anions, respectively. Lipo 20:0 BPD-PC generated the highest, while LNP BPD-Cholesterol generated the lowest singlet oxygen from the panel of V-LNPs. Lipo 20:0 BPD-PC produced 2.07-fold higher singlet oxygen compared to LNP BPD-Cholesterol when measured using the SOSG probe. Interestingly, LNP BPD-Cholesterol produced the highest and Lipo 20:0 BPD-PC produced the lowest hydroxyl radicals and peroxynitrite anions. LNP BPD-Cholesterol was able to produce 4.07-fold higher hydroxyl radicals and peroxynitrite anions than Lipo 20:0 BPD-PC. As such, an opposite trend in the generation of Type II singlet oxygen and Type I hydroxyl radical and peroxynitrite anion generation was observed between Lipo 20:0 BPD-PC and LNP BPD-Cholesterol. A potential switch from Type II to Type I photochemistry for two different formulations (LNP 20:0 BPD-PC and Lipo BPD-PC) of the same photosensitizer has been reported. A similar transition from Type II to Type I photochemistry using a micelle formulation of photosensitizer 5,10,15,20-tetrakis(meso-hydroxyphenyl) porphyrin (mTHPP) has also been reported. This shift in photochemistry is likely because of the presence of the electron rich poly(2-(diisopropylamino)ethyl methacrylate) (PDPA) in the environment. This shift in photochemistry is likely attributed to the presence of the ionizable lipid SM-102 at the core of V-LNPs which may function as electron rich molecule.

Determining subcellular localization of the verteporfin-lipid nanoparticles: Subcellular localization is critical in PDT, as it determines the specific organelles targeted for ROS production, influencing PDT outcomes. An endoplasmic reticulum (ER) targeting photosensitizer designed to enhance PDT outcomes (PhotoOx) localizes in the ER which significantly increases intracellular ROS production and amplifies ICD-related tumor immunogenicity in 4T1 murine breast cancer cells. To further investigate the impact of subcellular localization on PDT efficacy, a series of Ce6-based small molecule conjugates were developed to modify intracellular distribution. These photosensitizers exhibited distinct organelle-specific accumulation in 4T1 murine breast cancer cells: Ce6 predominantly localized to the ER, LS765 and LS897 targeted lysosomes, while LS909 was distributed across both lysosomes and mitochondria. Previous reports have highlighted that free BPD localizes to the ER/mitochondria, triggering apoptotic pathways, whereas a liposomal formulation of lipidated BPD (20:0 BPD-PC and 16:0 BPD-PC) predominantly accumulates in lysosomes, potentially leading to lysosomal disruption and autophagic cell death. Understanding these differences is essential for optimizing PDT efficacy and designing nanoformulations. Therefore the intracellular localization of V-LNPs in CT1BA5 cells was examined using confocal microscopy. Cells were incubated with LNP 20:0 BPD-PC, LNP 16:0 BPD-PC, LNP BPD-Cholesterol, LNP Free BPD, and Lipo 20:0 BPD-PC for 24 h. After incubation, staining of the CT1BA5 cells was performed with Lyso-Tracker Green DND-26, Mito-Tracker, and ER-Tracker to visualize lysosomes, mitochondria, and the ER, respectively. The intrinsic fluorescence of BPD, 20:0 BPD-PC, 16:0 BPD-PC, and BPD-Cholesterol was utilized for imaging.

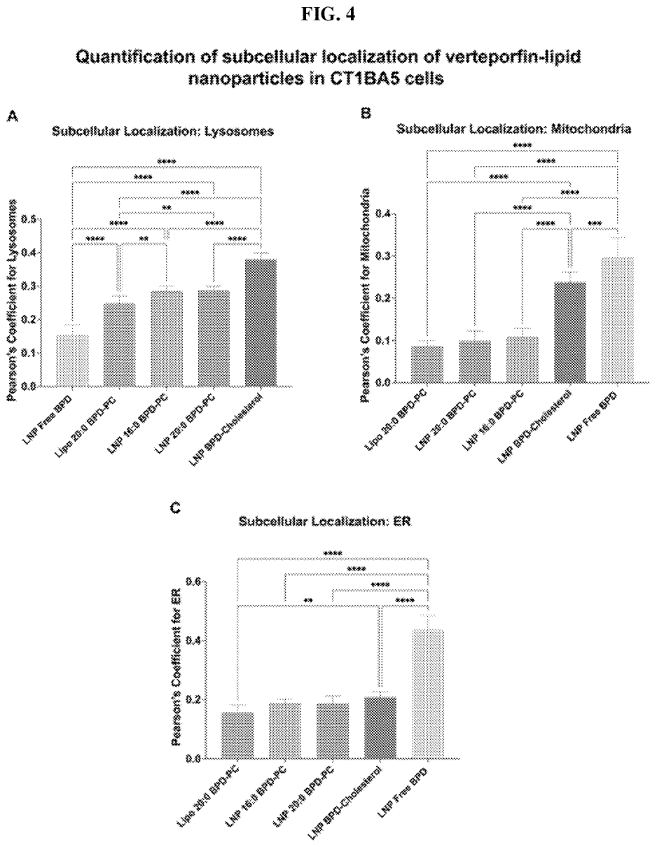

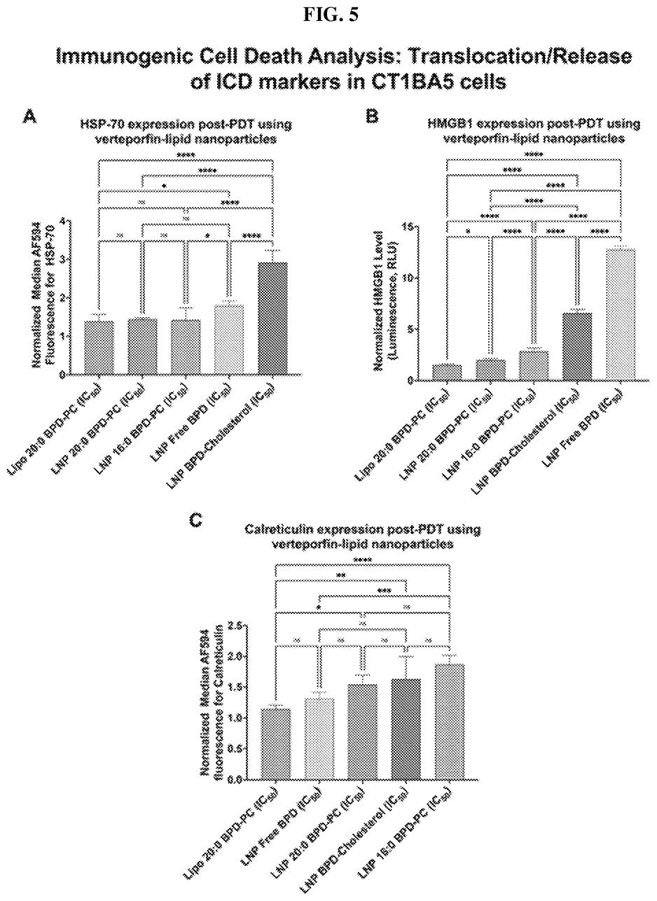

FIG. 4 shows quantification of Pearson's coefficient values for co-localized formulations in CT1BA5 cells within (A) Lysosomes, (B) Mitochondria, and (C) Endoplasmic reticulum (ER). All data are presented as mean±S.D., statistical significance was calculated using a one-way ANOVA test on GraphPad Prism v10.4.1, *: P<0.1, **: P<0.01, ***: P<0.001, *****: P<0.0001. As shown in FIG. 4, Lipo 20:0 BPD-PC, LNP 20:0 BPD-PC, and LNP 16:0 BPD-PC exhibited predominant localization within lysosomes, with Pearson's correlation coefficients of 0.25, 0.29, and 0.28, respectively, confirming strong lysosomal accumulation. LNP BPD-Cholesterol showed dual localization in lysosomes and mitochondria, with Pearson's correlation coefficient values of 0.38 for lysosomes and 0.24 for mitochondria. Further, LNP Free BPD showed the highest accumulation in ER and mitochondria with Pearson's correlation coefficients of 0.43 and 0.29, respectively. Representative confocal microscopy images of CT1BA5 cells imaged using a 100× objective after a 24 h incubation with all the V-LNPs were visualized using three different fluorescent trackers. Overall, these findings align with previous reports, where free BPD localizes in the ER and mitochondria, while 20:0 BPD-PC and 16:0 BPD-PC localize in lysosomes. Most interestingly, BPD-Cholesterol localizes in both mitochondria and lysosomes, indicating a distinct localization pattern that could influence PDT outcomes. This suggests the fine tuning of photosensitizers to target specific subcellular organelles is critical for enhancing PDT efficacy.

Measuring the light induced phototoxicity of verteporfin-lipid nanoparticles: The effects of PDT using LNP 16:0 BPD-PC, LNP BPD-Cholesterol, and LNP Free BPD with and without light on the metabolic activity of CT1BA5 and 6620c1 cells was examined. These results were compared to previously reported metabolic activity of LNP 20:0 BPD-PC and Lipo 20:0 BPD-PC. All the LNP formulations exhibited dark toxicity, leading to a decrease in the metabolic activity of CT1BA5 cells. This effect may be attributed to the presence of the ionizable lipid SM-102 which is used in LNP synthesis. Previous studies have reported similar cytotoxic effects using ionizable lipids like D-Lin-MC3-DMA, in HEK-293T cells. LNP Free BPD and LNP BPD-Cholesterol demonstrated the highest decrease in metabolic activity of CT1BA5 cells, followed by LNP 20:0 BPD-PC and Lipo 20:0 BPD-PC. LNP 16:0 BPD-PC demonstrated the lowest decrease in metabolic activity of CT1BA5 cells among the panel of V-LNPs used. This reduction in metabolic activity can be attributed to higher generation of hydroxyl radical and peroxynitrite anion generation and better cellular uptake by the respective formulations. A similar phototoxicity trend was observed in 6620c1 cells. The IC₂₅, IC₅₀, and IC₇₅ values for each formulation were extrapolated from the cell viability curve which was obtained using an MTT assay. These values are presented below in Table 2 for CT1BA5 cells and Table 3 for 6620c1 cells. Table 2 shows a summary of BPD equivalent inhibitory concentrations of a panel of V-LNPs in CT1BA5 cells activated using a fluence of 20 J/cm² with 690 nm light (irradiance of 17.86 mW/cm²). Results were obtained from the MTT assay. Values are mean, n=6+/−SD. Table 3 shows a summary of BPD equivalent inhibitory concentrations of a panel of V-LNPs in 6620c1 cells activated using a fluence of 20 J/cm² of 690 nm light (irradiance of 17.86 mW/cm²). Results were obtained from the MTT assay. Values are mean, n=6+/−SD.

TABLE 2

| Lipid-based | IC₂₅ | IC₅₀ | IC₇₅ |
|---|---|---|---|
| Nanoformulation | (nM ± S.D.) | (nM ± S.D.) | (nM ± S.D.) |
| LNP Free BPD | 7.2 ± 0.9 | 13.9 ± 2.9 | 27.1 ± 4.2 |
| LNP BPD-Cholesterol | 5.9 ± 2.1 | 14.8 ± 2.6 | 30.3 ± 3.2 |
| LNP 20:0 BPD-PC | 11.4 ± 1.9 | 26.2 ± 2.1 | 49.7 ± 3.6 |
| Lipo 20:0 BPD-PC | 18.8 ± 2.8 | 39.9 ± 3.3 | 77.8 ± 6.5 |
| LNP 16:0 BPD-PC | 25.3 ± 3.8 | 49.0 ± 4.3 | 90.8 ± 12.7 |

TABLE 3

| Lipid-based | IC₂₅ | IC₅₀ | IC₇₅ |
|---|---|---|---|
| Nanoformulation | (nM ± S.D.) | (nM ± S.D.) | (nM ± S.D.) |
| LNP Free BPD | 8 ± 1.9 | 21 ± 3.8 | 43 ± 5.3 |
| LNP BPD-Cholesterol | 13 ± 1.2 | 33 ± 2.9 | 69 ± 6.0 |
| LNP 20:0 BPD-PC | 31 ± 4.7 | 103 ± 9.1 | 227 ± 13.6 |
| LNP 16:0 BPD-PC | 36 ± 3.2 | 100 ± 6.1 | 207 ± 16.5 |
| Lipo 20:0 BPD-PC | 94 ± 4.6 | 222 ± 14.2 | 447 ± 22.5 |

The uptake of free and lipidated BPD in CT1BA5 cells using LNP 16:0 BPD-PC, LNP 20:0 BPD-PC, LNP BPD-Cholesterol, LNP Free BPD, and Lipo 20:0 BPD-PC as delivery vehicles was measured. LNP Free BPD (2.53×10⁻⁷ nanomoles of BPD equivalent concentration per cell) had the highest accumulation in CT1BA5 cells, followed by LNP BPD-Cholesterol (2.41×10⁻⁷ nanomoles of BPD equivalent concentration per cell). The remaining constructs ranged from 5.80×10⁻⁸ to 1.15×10⁻⁷ nanomoles of BPD equivalent concentration per cell. Lipo 20:0 BPD-PC showed the lowest uptake in CT1BA5 cells. Generally speaking, the trends in phototoxicity levels corresponded to the cellular uptake levels of the V-LNPs.

Measuring PDT-induced immunogenic cell death: As mentioned earlier, PDT induces ICD through the release/translocation of DAMPs such as HSP-60, HSP-70, calreticulin, HMGB1, and extracellular ATP in dying or stressed cells. Additionally, these DAMPs activate macrophages and dendritic cells, which further activate T-cell mediated adaptive immunity through tumor-specific antigens. Normally, calreticulin is normally expressed intracellularly in the ER lumen. In a stressed condition or during ICD, calreticulin translocates to the surface of the cell membrane to send “eat me” signals to phagocytic cells such as macrophages and dendritic cells to induce a T-cell based immune response. HSPs are generally regarded as chaperones that help in protein folding and translocation. Among them, HSP-70 has been studied as an ICD marker post-PDT. HSP-70 is known to translocate to the cell membrane during ICD. Literature has demonstrated that BPD based lipid formulations can effectively induce ICD. The ability to induce ICD using a triple-targeted liposomal formulation of BPD in a 3D pancreatic cancer model has been reported using 690 nm light irradiation with a fluence up to 100 J/cm² at an irradiance of 100 mW/cm. The ability of liposomal BPD to induce ICD in pancreatic and head and neck tumors has also been shown. The ICD-inducing potential of Lipo 20:0 BPD-PC and LNP 20:0 BPD-PC by measuring calreticulin exposure using IC₂₅ and IC₅₀ BPD-PC equivalent concentrations following 690 nm light irradiation has also been investigated. Results demonstrated that LNP 20:0 BPD-PC was more efficient at inducing ICD at a lower PDT dose.

The role of different V-LNPs in inducing ICD by measuring the exposure of different ICD markers (calreticulin, HSP-70, and HMGB1) post-PDT in CT1BA5 cells was studied. The release/translocation of calreticulin, HSP-70, and HMGB1 on/from the cell surface was measured using three different BPD equivalent inhibitory concentrations (IC₂₅, IC₅₀, and IC₇₅) which were obtained from a metabolic activity assay for each formulation post-irradiation by 690 nm light using a fluence of 20 J/cm² at an irradiance of 17.86 mW/cm² in CT1BA5 cells. Results demonstrate that with an increase in PDT dose, there is an increase in the exposure of calreticulin, HSP-70, and HMGB1 signals. For calreticulin signals, LNP Free BPD showed the highest increase, followed by LNP BPD-Cholesterol. Meanwhile, Lipo 20:0 BPD-PC demonstrated the lowest increase in calreticulin signals.

FIG. 5 shows exposure of the immunogenic cell death markers (A) HSP-70, (B) HMGB1 and (C) Calreticulin in CT1BA5 cells post PDT using 690 nm light with a fluence of 20 J/cm² and IC₅₀ BPD equivalent concentration of a panel of V-LNPs. All data are presented as mean±S.D., statistical significance was calculated using a one-way ANOVA test on GraphPad Prism v10.4.1, *: P<0.1, **: P<0.01, ***: P<0.001, ****: P<0.0001. LNP BPD-Cholesterol exhibited the highest increase in HSP-70 signals, followed by LNP Free BPD while LNP 20:0 BPD-PC and Lipo 20:0 BPD-PC showed the lowest increase in HSP-70 signals. For the HMGB1 marker, similar trends were observed where LNP BPD-Cholesterol and LNP Free BPD showed the highest release of HMGB1 while Lipo 20:0 BPD-PC demonstrated the lowest release of HMGB1 post-PDT. The role of different V-LNPs in inducing ICD by measuring the exposure of different ICD markers post-PDT in 6620c1 cells was examined. The exposure of HSP-70, HMGB1, and calreticulin using the IC₅₀ BPD equivalent concentration of a panel of V-LNPs post-PDT in 6620c1 cells was obtained. BPD-Cholesterol and LNP Free BPD exhibited the highest release of HMGB1 and HSP-70, consistent with trends observed in CT1BA5 cells, indicating that this response is not cell-line dependent. Overall, these findings demonstrates that Type I ROS production is associated with higher exposure of ICD markers like HMGB1 and HSP-70.

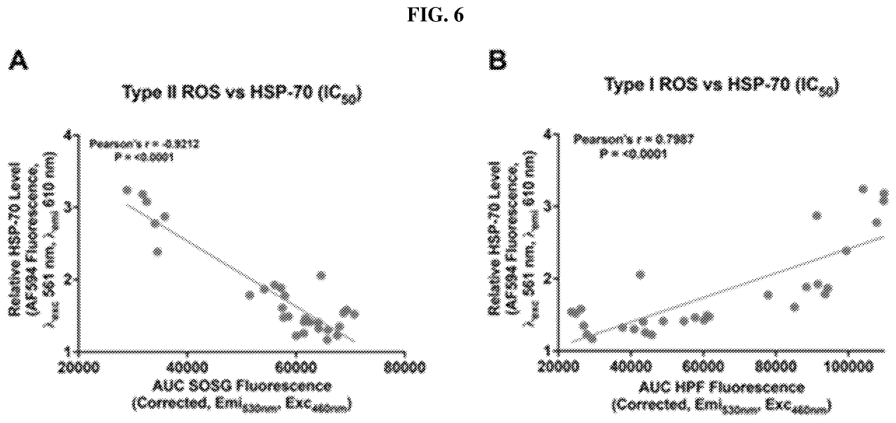

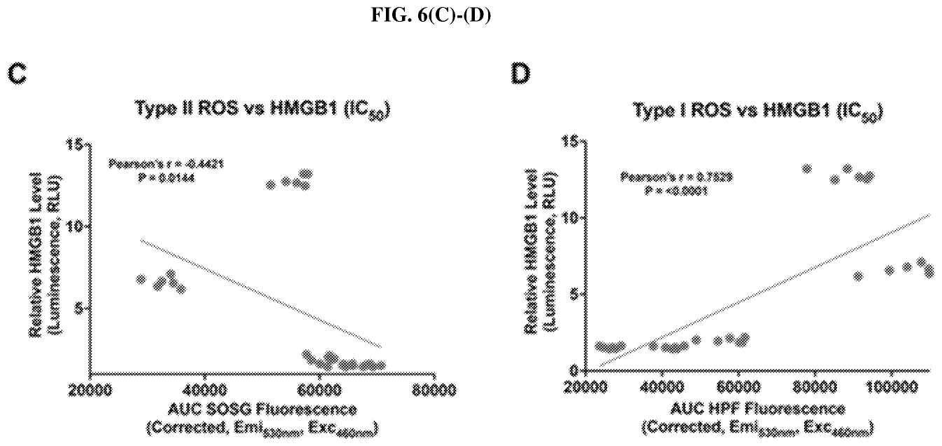

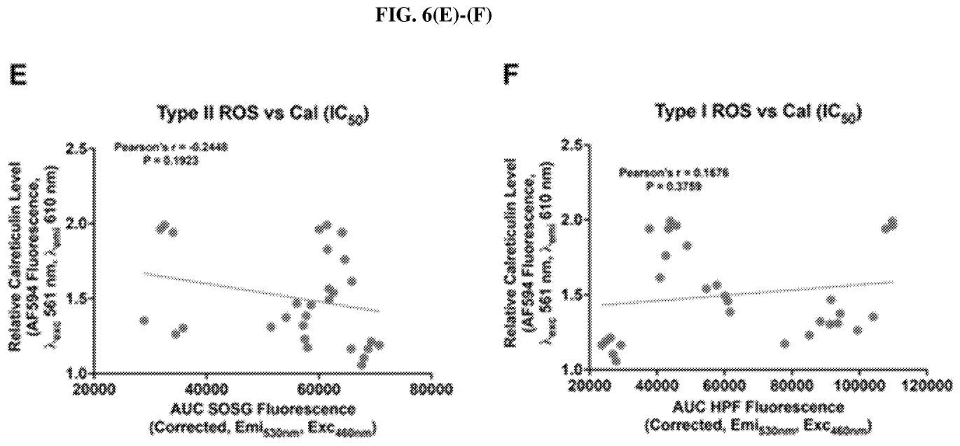

Investigating the relationships between the attributes of verteporfin-lipid nanoparticle (ROS and subcellular localization) and ICD: We performed a correlation analysis between the exposure of the ICD markers (HSP-70, HMGB1, and calreticulin) measured using three different BPD equivalent concentrations (IC₂₅, IC₅₀, and IC₇₅) of V-LNPs in CT1BA5 cells and the production of Type I and Type II ROS. FIG. 6 shows representative scatter plots showing the relationships between the exposure of the immunogenic cell death marker HSP-70, HMGB-1, and calreticulin at IC₅₀ BPD equivalent concentrations using different LNP and liposomal formulations and Type II ROS (A,C,E) and Type I ROS (B,D,F). HSP-70 and calreticulin were measured using flow cytometry and HMGB1 was measured using a bioluminescence kit (Promega). Type II is represented of AUC with SOSG probe and Type I is represented by AUC of hydroxyl radical and peroxynitrite anion measured using HPF probe. A statistically significant inverse correlation was observed between Type II ROS and HSP-70 exposure using the IC₂₅ (r=−0.5912, P=0.0006), IC₅₀ (r=−0.9212, P=<0.0001), and IC₇₅ (r=−0.7272, P=<0.0001) BPD equivalent concentrations of V-LNPs. Furthermore, a statistically significant direct correlation was observed between Type I ROS and HSP-70 exposure with IC₂₅ (r=0.6527, P<0.0001), IC₅₀ (r=0.7987, P=<0.0001) and IC₇₅ (r=0.7865, P=<0.0001) of V-LNPs. These correlations suggest that a higher production of Type I ROS rather, than Type II ROS, is required for better induction of ICD marker HSP-70 in CT1BA5 cells.

Similarly, a statistically significant inverse correlation was observed between Type II ROS and HMGB1 exposure using the IC₂₅ (r=−0.6079, P=0.0004), IC₅₀ (r=−0.4421, P=0.0144) and IC₇₅ (r=−0.5804, P=0.0008) BPD equivalent concentrations of V-LNPs. Furthermore, a statistically significant direct correlation was observed between Type I ROS and HMGB1 exposure with IC₂₅ (r=0.8252, P<0.0001), IC₅₀ (r=0.7529, P=<0.0001) and IC₇₅ (r=0.7958, P=<0.0001)) BPD equivalent concentrations of V-LNPs. These correlations suggest that a higher production of Type I ROS, rather than Type II ROS, is required for better induction of the ICD marker HMGB1 in CT1BA5 cells.

A correlation analysis was performed using calreticulin exposure. No statistically significant inverse correlation was observed between Type II ROS and calreticulin exposure using the IC₂₅ (r=−0.3863, P=0.0305), IC₅₀ (r=−0.2448, P=0.1923), and IC₇₅ (r=0.05371, P=0.7780) BPD equivalent concentrations of V-LNPs. Furthermore, no statistically significant direct correlation was observed between the Type I ROS and the calreticulin exposure using the IC₂₅ (r=0.3089, P=0.0967), IC₅₀ (r=0.1676, P=0.3759), and IC₇₅ (r=0.2065, P=0.1723) BPD equivalent concentrations of V-LNPs. It is believed that PDT using V-LNPs induces ICD through ferroptosis, which does not strongly involve calreticulin exposure.

To further confirm the relationship between the exposure of ICD markers and the type of ROS production by V-LNPs, a correlation analysis using 6620c1 cells was performed. A representative scatter plot illustrated the relationship between ICD markers and the type of ROS using IC₅₀ BPD equivalent concentration of the V-LNPs. A statistically significant inverse correlation was observed between Type II ROS and the exposure of ICD markers HSP-70 and HMGB1 using the IC₅₀ BPD equivalent concentration of V-LNPs. Furthermore, a statistically significant direct correlation was observed between Type I ROS and the exposure of ICD markers HSP-70 and HMGB1 using the IC₅₀ BPD equivalent concentration of V-LNPs. These findings are consistent with observations in CT1BA5 cells.

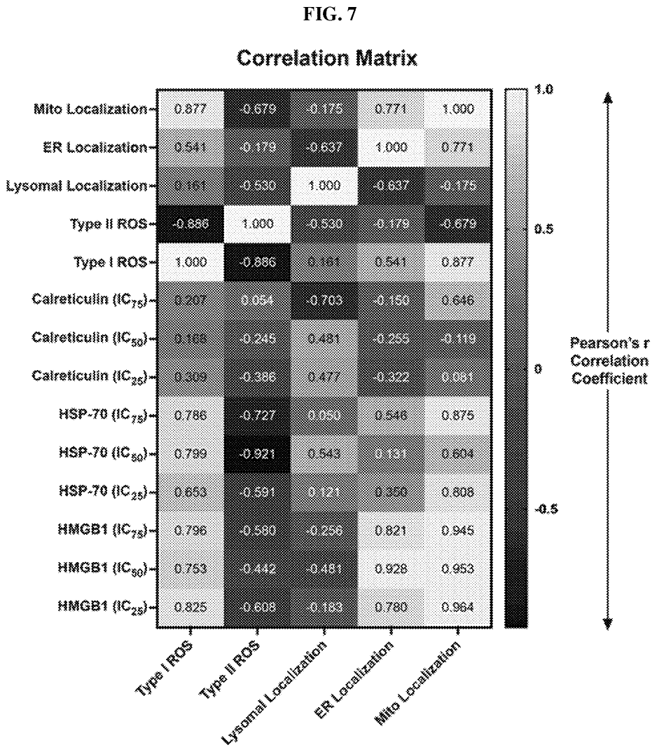

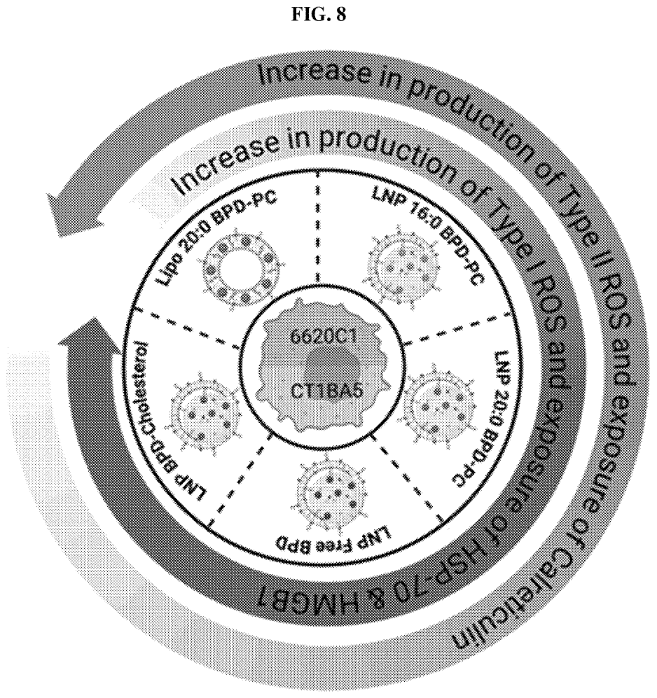

The relationships between the type of ROS species, subcellular localization, and PDT-induced ICD using IC₂₅, IC₅₀, and IC₇₅ doses of V-LNPs are summarized in a correlation matrix heat map in FIG. 7. FIG. 7 shows a heat map showing the Pearson's Coefficient matrix depicting the relationships between ROS generation (Type I and Type II), ICD markers (calreticulin, HSP-70, and HMGB1), and sub-cellular localization (lysosomes, mitochondria (Mito) and ER) for preferred embodiments of the V-LNPs. Heat map analysis was conducted using GraphPad Prism v10.4.1. The strongest direct correlations were observed between Type I ROS and the exposure of HSP-70, Type I ROS and the exposure of HMGB1, and mitochondrial localization and the exposure of HSP-70 and HMGB1. These findings suggest that increased Type I ROS production is strongly associated with higher HMGB1 and HSP-70 exposure. Additionally, higher mitochondrial localization of formulations correlates strongly with HMGB1 exposure indicating that mitochondria may play a crucial role in regulating ICD marker exposure. Similarly, ER localization exhibited a strong positive correlation with HMGB1, indicating that ER-associated photosensitizer accumulation may contribute to enhanced HMGB1 release. In contrast, a negative correlation was observed between Type II ROS and the exposure of both the markers HSP-70 and HMGB1, implying that higher Type II ROS does not contribute to the induction of these markers. Furthermore, mitochondrial localization displayed a weaker correlation with calreticulin exposure, suggesting that increased mitochondrial accumulation may be less favorable for calreticulin exposure. Overall, as shown in FIG. 8 these findings suggest that Type I ROS is more strongly linked to the induction of ICD markers such as HSP-70 and HMGB1. Additionally, the localization of formulations plays a critical role in modulating these relationships, potentially impacting their therapeutic efficacy in PDT-mediated immune activation.

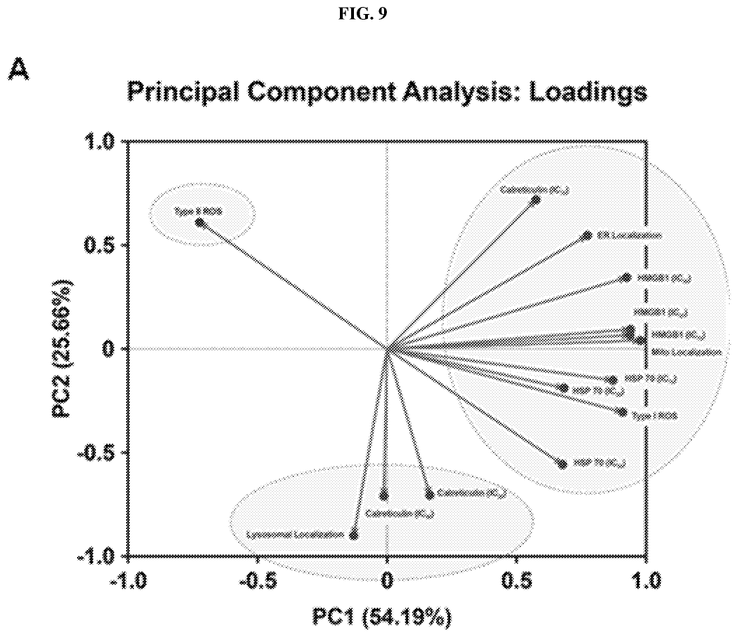

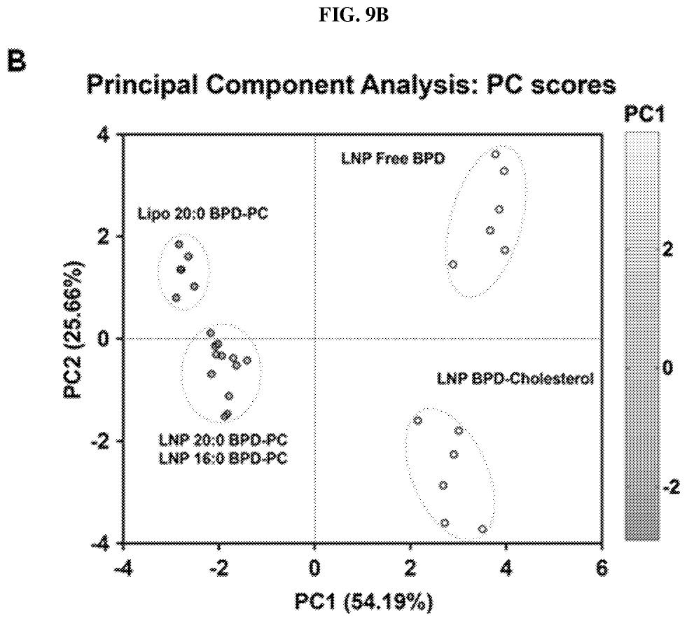

Principal Component Analysis to determine the multi-variate relationships between verteporfin-lipid nanoparticles attributes and ICD: PCA was conducted to assess the contribution of different principal components (PCs) to the overall variance in the dataset. The dataset included different attributes and loadings. The proportion of variance explained by each PC, both individually and cumulatively, was considered. FIG. 9 shows Principal Component Analysis (PCA) results, including (A) PC scores plot showing sample distribution along the principal components, highlighting clustering patterns and group separations, and (B) PCA loadings plot illustrating the contribution of individual variables to each principal component, indicating important components driving variability in the dataset. PCA analysis was conducted using GraphPad Prism v10.4.1. The first two PCs (PC1 and PC2) were selected based on their cumulative energies, which accounted for 79.84% of the total energies. Individually, PC1 and PC2 accounted for 54.19% and 25.66% of the total energies, respectively. Subsequent components contributed less, suggesting that PC1 and PC2 capture the most relevant data structure. The proportion of variance explained by each component was visualized through a bar graph, where the cumulative energy of each PC is represented by separate bars, and the individual contribution is shown as a line graph. The decline in energy contribution after PC2 supports our selection of the first two PC for further analysis.

The loading plot illustrates the contribution of each variable to the first two PCs (PC1 and PC2). The loading plot identifies relationships among variables, where positively correlated variables are positioned closely together while negatively correlated variables are plotted on opposite sides of the plot. As shown in FIG. 9(A) three distinct clusters were seen across both components. The first cluster consisted of Type II ROS, while the second cluster, positioned on the opposite side, included variables like Type I ROS, exposure of HMGB1 and HSP-70 (IC₂₅, IC₅₀, and IC₇₅), and mitochondrial and ER localization. The second cluster exhibited a strong contribution to PC1, with moderate to weak association with PC2. This data suggests a positive correlation among the variables of the second cluster while indicating a negative correlation with Type II ROS in the first cluster. A third cluster was observed between the first and second clusters along PC1, including variables like lysosomal localization and calreticulin exposure (IC₂₅ and IC₅₀). Interestingly, calreticulin (IC₇₅) exposure clustered within the second cluster, suggesting that at higher concentrations, calreticulin exposure no longer positively correlates with lysosomal localization. This shift likely indicates a concentration-dependent difference in the exposure of calreticulin. Overall, the results demonstrate that it is important to have higher Type I ROS production and mitochondria and ER localization of photosensitizer for enhanced ICD (HMGB1 and HSP-70) response.

PC scores are numerical values that define the positioning of each sample along the PC, offering a simplified representation of complex datasets. Each sample is assigned a PC score for each PC, which determines its placement in the PCA plot. As shown in FIG. 9(B), distinct clustering patterns were observed: LNP Free BPD and LNP BPD-Cholesterol clustered on the positive side of PC1, indicating similarities in their ROS generation, subcellular localization, and PDT-induced ICD. LNP 20:0 BPD-PC and LNP 16:0 BPD-PC clustered together at negative PC1 and near zero PC2, suggesting similarities in lipid composition, type of ROS production, and subcellular localization. Lipo 20:0 BPD-PC clustered toward the positive PC2 component, representing an opposite behavior compared to the other formulations. The clustering of LNP BPD-Cholesterol and LNP Free BPD in the positive PC1 region aligns with their higher Type I ROS production, which correlates with enhanced HSP-70 and HMGB1 exposure.

In summary, in this example, a panel of V-LNPs containing unmodified verteporfin or verteporfin conjugated to cholesterol, 20:0 lyso PC, or 16:0 lyso PC was synthesized and characterized. Their photophysical and photochemical properties, PDT efficacy, and their ability to induce ICD were examined. V-LNPs varied significantly in generation of type of ROS, subcellular localization and exposure of ICD in CT1BA5 cells. A significant direct correlation was shown between Type I ROS and the exposure of ICD markers HMGB1 and HSP-70 in CT1BA5 and 6620c1 cells, while an inverse relationship was observed between HMGB1 and HSP-70 exposure and Type II ROS. Furthermore, for the first time, a machine learning approach (PCA) was utilized to understand the interrelationship between ROS types, subcellular localization, and ICD. PCA data demonstrated a strong contribution of Type I ROS and mitochondrial/ER localization-related variables to PC1 highlighting their role in enhancing ICD marker exposure, while Type II ROS contributes the least to the exposure of ICD markers. Overall, these results demonstrate that fine-tuning ROS production and subcellular localization can enhance in vitro tumor immune responses through ICD. These effects depend on factors such as the nature of verteporfin conjugates, the type of photosensitizers, lipid-based nanoformulations, non-pancreatic cancer cell lines, their relationship to cell death mechanisms, the role of hypoxia-activated sensitizers, and how ICD translates to T-cell responses.

### Example 2

This example analyzes the impact of incorporating the clinically approved ionizable lipid SM-102 into light-activated liposomes containing the verteporfin-lipid conjugate 20:0 BPD-PC on photochemical ICD of CT1BA5 mouse pancreatic cancer cells. SM-102 incorporation increases the ratio of Type I (hydroxyl radical and peroxynitrite anion) to Type II (singlet oxygen) reactive oxygen species by ~2.5-fold, improves re-localization of the verteporfin-lipid conjugate 20:0 BPD-PC to the mitochondria, and improves cancer cell uptake and phototoxicity by up to 8-fold. This results in a 2-3-fold increase exposure of the DAMPs HSP-70, HMBG1 and Calreticuling following activation with 690 nm light without increasing dark toxicity. This leads to a significant improvement in CT1BA5 tumor growth inhibition in an immunocompetent mouse model.

ICD is critical for driving effective antitumor immunity, making it important to design and tailor PDT formulations that maximize ICD induction. This is especially relevant for cancers that respond poorly to immune checkpoint inhibitors, such as pancreatic ductal adenocarcinoma (PDAC), where overall response rates are typically less than 5%. These negligible responses are largely attributed to poor T-cell infiltration, low immunogenicity, low microsatellite instability, and an immunosuppressive microenvironment. Some PDT-based approaches that enhance ICD are promising strategies for converting immunologically “cold” tumors into “hot” and potentially improve responses to immune checkpoint inhibitors. Solid LNPs containing the clinical ionizable lipids SM-102 and the lipid-anchored photosensitizer BPD-PC were more efficient at inducing ICD than a conventional liposomal formulation of BPD-PC. Liposomes assume well-defined spherical lipid bilayered structures with an aqueous core upon hydration at temperatures exceeding the lipid melting temperatures, while LNPs assume spherical non-lamellar structures with amorphous solid lipid cores following synthesis using nano-precipitation below the lipid melting temperatures. As such, unlike liposomes, solid LNPs have no capacity to encapsulate aqueous drug payloads, and play a limited role in combination therapies using encapsulated aqueous agents. A targeted photoactivable multi-inhibitor liposome (TPMIL) co-encapsulating the hydrophobic photosensitizer 20:0 BPD-PC and the hydrophilic chemotherapy agent irinotecan exhibited superior antitumor efficacy when used in combination with PDT and significantly de-escalated chemotherapy doses. We have also shown that liposomes containing BPD-PC are amenable to targeting single and multiple tumor receptors, which leads to enhanced therapeutic outcomes when used to co-encapsulate the chemotherapy agents gemcitabine hydrochloride, 5-fluorouracil, and oxaliplatin. Demonstrating their amenability for both hydrophilic and hydrophobic agent encapsulation, liposomes co-loaded with hydrophobic gold nanoclusters, hydrophobic BPD, and hydrophilic oxaliplatin enhanced the therapeutic response to radiation therapy in pancreatic cancer models.

In this example, the clinical ionizable lipid SM-102 (a component of the SPIKEVAX LNP vaccine) was doped into light activatable BPD-PC liposomes and its impact on ICD and treatment response was investigated. Based on the findings of the unsupervised machine learning described above in the data-driven process, the role of SM-102 incorporation into liposomes on photochemical radical production, intracellular uptake efficiency, and mitochondria/ER localization was explored. Both of these are key features of light activated lipid nanoformulations that are associated with photochemical ICD. This establishes a foundation for combination strategies using liposomes that have enhanced ICD responses by simply incorporating the clinical ionizable lipid SM-102.

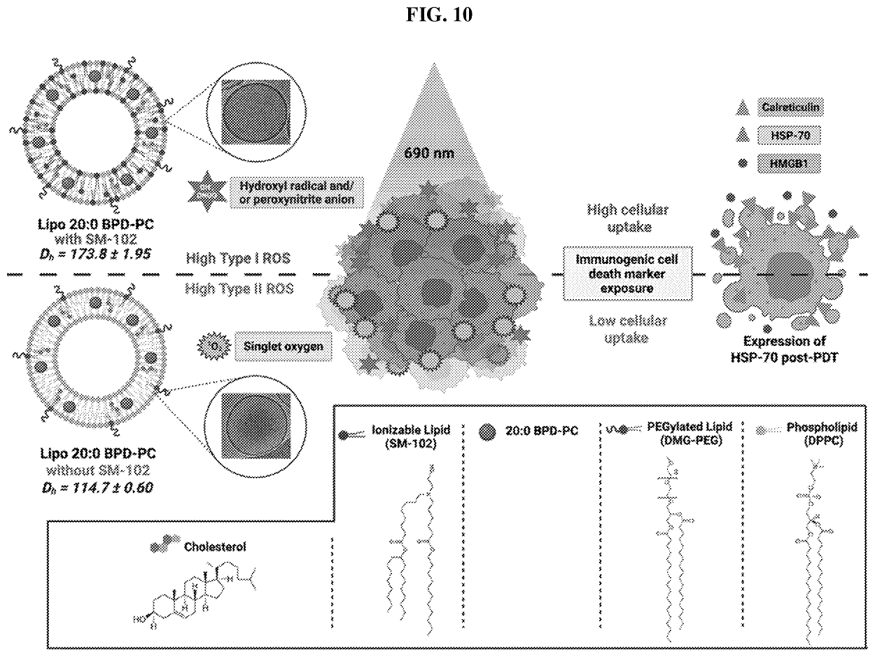

FIG. 10 shows a graphical representation of PDT-induced immunogenic cell death (HSP-70, calreticulin, and HMGB1 exposure) using Lipo 20:0 BPD-PC (SM-102) and Lipo 20:0 BPD-PC (without SM-102) in murine pancreatic cancer cells that produce varying levels of Type I and Type II ROS, localize differentially to the mitochondria, and exhibit varying degrees of phototoxicity.

### Materials and Methods

Conjugation of BPD to 20:0 lyso PC: To synthesize the verteporfin lipid conjugate (20:0 BPD-PC), BPD was conjugated to 1-arachidoyl-2-hydroxy-sn-glycero-3-phosphatidylcholine (20:0 lyso PC). Briefly, 20:0 lyso PC (Avanti Polar Lipids), BPD (US Pharmacopeia), 1-ethyl-3-(3-dimethylaminopropyl) carbodiimide (EDC; Sigma-Aldrich), 4-(dimethylamino) pyridine (DMAP; Sigma-Aldrich), and N,N-diisopropylethylamine (DIPEA; Sigma-Aldrich) were combined in a molar ratio of 1:5:50:25:60, respectively, in 5 mL of ethyl acetate. The reaction mixture was vortexed briefly at 2000 rpm and stirred at 2500 rpm for 72 h, following which ethyl acetate was removed from the reaction mixture using a gentle stream of nitrogen gas. The dried reaction mixture residue was then redissolved in 1 mL of a mobile phase consisting of chloroform, methanol, and water (65:25:1, v/v/v). A silica gel column was packed by mixing Silica Gel 60 (Sigma-Aldrich) with 10 mL of the mobile phase. The redissolved mixture was transferred to a chromatography column (Kemtech America Inc.) and conditioned with 10 mL of the mobile phase prior to loading the sample. The reconstituted reaction mixture was loaded onto the silica column, followed by elution with an additional 15 mL of the mobile phase. The slowest fraction containing the conjugated product (20:0 BPD-PC) was collected and concentrated by rotary evaporation at a temperature of 30° C. The purified 20:0 BPD-PC residue was redissolved in 1 mL of chloroform, filtered through a 0.22 μm syringe filter (VWR), and dried again by rotary evaporation at 30° C. An additional four chloroform washes (total of 5 mL) were performed to collect the remaining 20:0 BPD-PC. The final purified 20:0 BPD-PC product was stored in a glass vial at −20° C. in the dark.

Synthesis of Lipo 20:0 BPD-PC with and without SM-102: For the preparation of Lipo 20:0 BPD-PC (without SM-102), 1,2-dipalmitoyl-sn-glycero-3-phosphatidylcholine (DPPC; Avanti Polar Lipids), 1,2-dimyristoyl-sn-glycero-3-phosphoethanolamine-N-[methoxy (polyethylene glycol)] (DMG-PEG₂₀₀₀; NOF America Corporation), cholesterol (Avanti), and 20:0 BPD-PC were combined in a 5 mL glass vial at a molar ratio of 59:1.5:38.5:1, respectively. For the Lipo 20:0 BPD-PC (SM-102) formulation, DPPC, DMG-PEG, cholesterol, the ionizable lipid 1-octylnonyl 8-{(2-hydroxyethyl)[6-oxo-6-(undecyloxy)hexyl]amino}octanoate (SM-102); BroadPharm), and 20:0 BPD-PC were combined at a molar ratio of 9:1.5:38.5:50:1, respectively, in a 5 mL glass vial. Both liposomal formulations were prepared using the conventional thin-film hydration method. The lipid mixtures were dissolved in chloroform, and the solvent was evaporated using a gentle stream of nitrogen gas to form a uniform lipid film. The dried films were hydrated with 1 mL of 1×DPBS (pH 7.4), heated in a water bath at 42° C. for 30 min, and subjected to ultrasonication using a probe tip sonicator (Fisher Scientific) for a total of 30 min (20 s on/40 s off cycles) at 42° C. in the dark. The resulting Lipo 20:0 BPD-PC formulations, with and without SM-102, were stored at 4° C. in the dark until further use.

Cryo-Transmission electron microscopy: For cryo-TEM, 3-4 μL of Lipo 20:0 BPD-PC (SM-102), Lipo 20:0 BPD-PC (without SM-102), suspension was applied onto 300-mesh Lacey carbon grids (Ted Pella, Inc.) that had been glow-discharged for 80 s at 30 mA. After a single blotting step of ~4 s with filter paper (Ted Pella, Inc.), the grids were vitrified by plunge-freezing into liquid ethane cooled by liquid nitrogen using a Leica GP2 automated plunge freezer (Leica Microsystems). Imaging was performed on either a Talos Arctica or Glacios transmission electron microscope (Thermo Fisher Scientific) operated at 200 kV and equipped with a K3 direct electron detector (Gatan). Images were collected in low-dose mode with SerialEM (v4.1 or newer) at a calibrated pixel size of approximately 1 Å/pixel. Each exposure (2.5 s total) consisted of 50 frames with an average electron dose of ~20 e-/pixel/s.

Formulation Characterization: The hydrodynamic diameters and ζ-potentials of Lipo 20:0 BPD-PC (SM-102) and Lipo 20:0 BPD-PC (without SM-102) were measured using a dynamic light scattering (DLS) system (Malvern Panalytical Inc). The BPD-PC equivalent concentration of each formulation was quantified by UV-Vis spectrophotometry (Thermo Scientific Evolution 350) using an extinction coefficient of ε₆₈₇ nm=34,895 M⁻¹ cm⁻¹ in dimethyl sulfoxide (DMSO; Sigma-Aldrich). UV-Visible absorbance spectra of intact formulations in DPBS were also measured as 5 μM BPD-PC equivalent dilutions of Lipo 20:0 BPD-PC (SM-102) and Lipo 20:0 BPD-PC (without SM-102).

Reactive oxygen species (ROS) generation: Singlet oxygen production was measured using the fluorometric probe Singlet Oxygen Sensor Green (SOSG; Fisher Scientific), and hydroxyl radical and/or peroxynitrite anion production was measured using hydroxyphenyl fluorescein (HPF; Fisher Scientific). For singlet oxygen measurements, 10 μL of 50 μM SOSG was added to 100 μL of each 5 μM BPD-PC equivalent formulation prepared in DPBS. For hydroxyl radical and/or peroxynitrite anion measurements, 20 μL of 200 μM HPF was added to 100 μL of each corresponding 5 μM BPD-PC equivalent sample in DPBS. All samples were irradiated with 690 nm LED light (Biolambda) using fluences of 0 J/cm², 0.5 J/cm², 1 J/cm², 1.5 J/cm², 2 J/cm², 2.5 J/cm², 3 J/cm², 3.5 J/cm², 4 J/cm², 4.5 J/cm², 5 J/cm², 10 J/cm², 15 J/cm², and 20 J/cm² at an irradiance of 17.86 mW/cm². Following irradiation, fluorescence intensities of SOSG and HPF were measured using a Tecan Spark plate reader (excitation: 460 nm; emission: 530 nm).

Cell culture: The CT1BA5 pancreatic cancer cell line was derived from KPfC (KrasLSL-G12D; Trp53f l/f l; PDXCre/+) mice established in the Brekken Lab. CT1BA5 cells were maintained in Dulbecco's Modified Eagle Medium (DMEM) with high glucose, supplemented with 10% fetal bovine serum (FBS), and 1× penicillin-streptomycin, and incubated at 37° C. in a humidified incubator with 5% CO₂.

Cellular uptake: 50,000 CT1BA5 cells per well were seeded in a white-walled, clear-bottom 96-well plate (Corning) and incubated at 37° C. for 24 h. Cells were then treated with 250 nM BPD-PC equivalent concentrations of Lipo 20:0 BPD-PC (SM-102) and Lipo 20:0 BPD-PC (without SM-102). Following a 24 h incubation, cells were washed three times with 100 μL of DPBS, and 100 μL of DPBS containing 1% Triton X-100 was added to each well to lyse the cells and release intracellular 20:0 BPD-PC. For quantification, a fluorescence standard curve for each formulation was generated by preparing serial dilutions of each formulation's BPD-PC equivalent (ranging from 250 nM to 0.25 nM) in DPBS containing 1% Triton X-100. Plates were covered with aluminum foil and placed on a shaker for 1 h at room temperature. The fluorescence emission intensities of intracellular 20:0 BPD-PC extracted using DPBS containing 1% Triton X-100 were measured using a Tecan Spark plate reader (excitation: 435 nm; emission: 698 nm) and concentrations of intracellular BPD-PC equivalent for each formulation were interpolating from the corresponding standard curves.

Determining subcellular localization: CT1BA5 pancreatic cancer cells were seeded in a 96-well transparent glass bottom black walled plate (Celvis) at a density of 50,000 per well and incubated at 37° C. for 24 h. After 24 h, culture media was replaced with fresh media containing Lipo 20:0 BPD-PC (SM-102) or Lipo 20:0 BPD-PC (without SM-102) at a BPD equivalent concentration of 2,000 nM. After 24 h incubation, cells were stained with organelle specific fluorescent probes by replacing the media content in each well fresh media containing either 1 μg/ml of Hoechst (nuclei tracker; Cell Signaling Technology), 50 nM of the Lyso-tracker (lysosome tracker; Cell Signaling Technology), 50 nM of the Mito-tracker (mitochondria tracker; Cell Signaling Technology), or 1 μM of the ER-tracker (endoplasmic reticulum tracker; Cell Signaling Technology). After 1 h of incubation in the dark at 37° C., cells were washed three times using fresh media prior to imaging to assess colocalization of the BPD variants with the organelle markers. Imaging was performed using an Olympus FV300RS Confocal Laser Scanning Microscope equipped with a 100× oil immersion objective. Excitation wavelengths included a 405 nm laser for Hoechst excitation, a 488 nm laser for Lysotracker excitation, a 568 nm laser for Mitotracker or ER tracker excitation, and a 647 nm laser for excitation of the BPD in Lipo 20:0 BPD-PC (SM-102) and Lipo 20:0 BPD-PC (without SM-102).

Metabolic Activity Measurements under Dark and Light Conditions: CT1BA5 cells were trypsinized and seeded in clear-bottom 96-well plates (Corning) at a density of 1,500 cells per well and incubated at 37° C. for 24 h. After incubation, the culture medium was removed, and cells were treated with varying concentrations of Lipo 20:0 BPD-PC (SM-102) and Lipo 20:0 BPD-PC (without SM-102). Following a 24 h incubation with the formulations, the cells were irradiated with 690 nm LED light (Biolambda) at a fluence of 20 J/cm² and an irradiance of 17.86 mW/cm². After irradiation, the cells were incubated for an additional 72 h. Cancer cell metabolic activity was then assessed by adding 100 μL of a 1:10 dilution of Alamar Blue (A50101, Thermo Fisher Scientific) in fresh culture medium to each well, followed by a 3 h incubation at 37° C. The fluorescence of the Alamar Blue Assay product was measured using a Tecan Spark plate reader (excitation: 560 nm; emission: 590 nm). Dark toxicity controls were performed in parallel by treating cells with the same concentrations of each formulation without light irradiation, and the cell metabolic activity was also assessed using the Alamar Blue Assay. Dose response curves were plotted on GraphPad Prism v10.4.1 and IC₂₅, IC₅₀ and IC₇₅ values were determined for each formulation following light activation.

Immunogenic cell death marker analysis: CT1BA5 cells were seeded at a density of 45,000 cells per well in 6-well plates and incubated at 37° C. for 24 h. Cells were then treated with media containing three BPD-PC equivalent inhibitory concentrations (IC₂₅, IC₅₀, or IC₇₅) of Lipo 20:0 BPD-PC (SM-102), Lipo 20:0 BPD-PC (without SM-102), or LNP 20:0 BPD-PC (SM-102) as a control. After 24 h of incubation, cells were irradiated using 690 nm LED light at a fluence of 20 J/cm² and an irradiance of 17.86 mW/cm². After another 24-h incubation following light irradiation, cells were harvested by trypsinization and transferred into individual Eppendorf tubes. To reduce non-specific binding of antibodies, cells were incubated with 50 μL of TruStain (0.5 mg/mL, diluted 1:5 in phosphate azide buffer (PAB); BioLegend, 101320) on ice in the dark for 15 min. Following blocking, cells were resuspended in 50 μL of PAB containing anti-HSP-70 antibody (20 μg/mL; 1:400 dilution; Cell Signaling Technology, clone 4872S) or anti-calreticulin antibody (0.406 μg/mL, 1:400 dilution in antibody dilution buffer, Cell Signaling Technology; clone D3E6) to detect cell surface HSP-70 or calreticulin exposure. Cells were incubated on ice in the dark for an additional 15 min. After primary antibody staining, cells were washed with PAB, resuspended in 50 μL of PAB containing AF594-conjugated secondary antibody (2 mg/mL; 1:500 dilution; Cell Signaling Technology, 8889S), and incubated on ice for 15 min in the dark. Cells were then washed once more in PAB and finally resuspended in 300 μL of PAB for flow cytometry analysis using (BD LSRFortessa™ X-20 cell analyzer). To determine the median AF594 fluorescence emission corresponding to HSP-70 or calreticulin levels, a 561 nm laser and a 610/20 nm bandpass detector was used.

Extracellular HMGB1 release was evaluated using the Lumit™ HMGB1 luminescence-based immunoassay (Promega). CT1BA5 cells were seeded at 20,000 cells per well in 96-well plates and allowed to incubate for 24 h at 37° C. Cells were then incubated with three BPD-PC equivalent inhibitory concentrations (IC₂₅, IC₅₀, or IC₇₅) of Lipo 20:0 BPD-PC (SM-102), Lipo 20:0 BPD-PC (without SM-102). After 24 h of incubation, cells were irradiated using 690 nm LED light at a fluence of 20 J/cm² and an irradiance of 17.86 mW/cm². A 5× antibody working solution containing Anti-hHMGB1 mAV-SmBIT and Anti-hHMGB1 mAB-LgBiT was added (20 μL) per well and incubated for 60-90 min. After incubation with antibody. 25 μL of a 1:20 dilution Lumit™ detection substrate was added in each well, followed by gentle mixing using a shaker at 300-500 rpm. The plate was then incubated for 3-5 min before measuring luminescence readings using a multi-plate reader.

Tumor Implantation, PDT, and Systemic Toxicity: All animal experiments were conducted in accordance with protocols approved by the Institutional Animal Care and Use Committee (IACUC; Protocol No. 19-10). Male C57BL/6 mice (6 weeks old, ~20 g) were purchased from The Jackson Laboratory and housed in the animal facility at the University of Texas at Dallas Vivarium in compliance with the National Institutes of Health Guide for the Care and Use of Laboratory Animals. One day prior to tumor implantation, hair on the left flank of each mouse was removed using Wahl professional animal hair clippers. CT1BA5 cells were cultured in Dulbecco's Modified Eagle Medium supplemented with 10% fetal bovine serum (FBS). When the cells reached approximately 80% confluency, they were detached using trypsin and collected in culture medium. The cell suspension was counted and washed three times with DPBS to remove residual medium and trypsin. Cells were then resuspended to obtain a concentration of 1×10⁶ cells in 20 μL of sterile DPBS and injected subcutaneously into the left flank of each mouse. Tumor growth was monitored after implantation using digital calipers. Tumor volume was calculated using the formula ab²/2, where a represents the length of the longest tumor dimension and b represents the length of the shorter dimension. Approximately 14 days after implantation, tumors reached a volume of 50-70 mm³ and animals were randomized into three treatment groups: (1) Control (n=4), (2) Lipo 20:0 BPD-PC (SM-102)+PDT (n=6), and (3) Lipo 20:0 BPD-PC (without SM-102) (n=6). Prior to PDT, mice were administered sustained-release meloxicam subcutaneously as a pre-emptive analgesic to minimize treatment-associated discomfort. Animals were then intravenously injected with formulations at a dose equivalent to 0.58 mg/kg BPD. Immediately following administration, tumors in the PDT group were irradiated with a 690 nm laser at a fluence of 150 J/cm² and an irradiance of 100 mW/cm². Following treatment, tumor volumes were measured every other day until mice reached the study endpoint, defined as tumor volume exceeding 2000 mm³ or natural death. Data were analyzed using GraphPad Prism version 10.6.1.

### Results and Discussion

Characterization: Using the thin-film hydration method at a temperature that exceeds the lipid melting temperatures of DPPC (42° C.) liposomes were prepared with well-defined bilayer membranes containing 20:0 BPD-PC and an aqueous core. As a control solid LNPs with hydrophobic amorphous solid lipid cores containing 20:0 BPD-PC were also prepared using a nano-precipitation technique at a temperature below the lipid melting temperature of DPPC (18° C.)

The hydrodynamic diameters, PDIs, and ζ-potentials of Lipo 20:0 BPD-PC (SM-102) and Lipo 20:0 BPD-PC were calculated. The hydrodynamic diameters of Lipo 20:0 BPD-PC (SM-102) and Lipo 20:0 BPD-PC (without SM-102) were 173.8 nm and 114.5 nm, respectively, which fall within the commonly reported range of 100-180 nm for clinically relevant lipid-based nanoparticles. The PDIs were all below 0.2, indicating that the formulations were relatively monodisperse. The ζ-potentials of Lipo 20:0 BPD-PC (SM-102) and Lipo 20:0 BPD-PC (without SM-102) were near neutral at −1.37 mV, and −0.51 mV respectively. This is expected because SM-102 is ionizable and remains uncharged in neutral saline solution but becomes ionized and cationic when the pH is lowered below its pKa of 6.5.

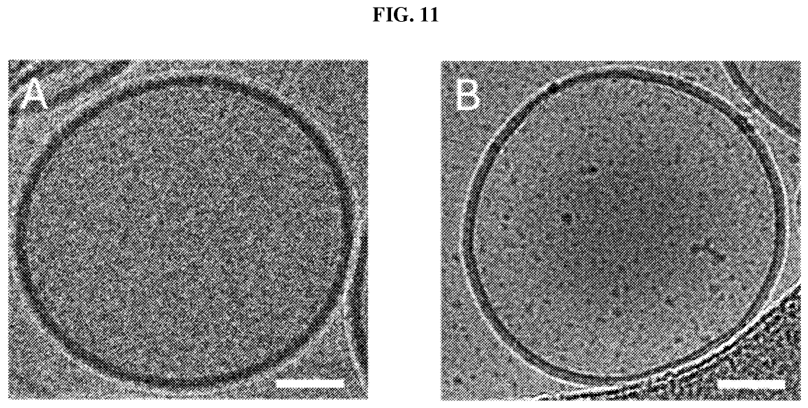

The raw absorbance spectra of Lipo 20:0 BPD-PC (SM-102) and Lipo 20:0 BPD-PC at a 5 μM BPD-PC equivalent concentration in DPBS shows the 690 nm absorbance peak at the Q-band maximum of 20:0 BPD-PC which was unaltered by the incorporation of SM-102 into the liposomes. The morphology of Lipo 20:0 BPD-PC (SM-102) and Lipo 20:0 BPD-PC (without SM-102) was examined using cryo-transmission electron microscopy (cryo-TEM). FIG. 11 shows representative cryo-Transmission Electron Microscopy (TEM) images of (A) Lipo 20:0 BPD-PC (with SM-102), (B) Lipo 20:0 BPD-PC (without SM-102). Scale bars are 100 nm. As shown in FIG. 11, the micrographs reveal that Lipo 20:0 BPD-PC formulations, whether they contain SM-102 or not, exhibit well-organized concentric bilayers enclosing an aqueous interior, consistent with the typical lamellar structure of conventional liposomes. These liposomes are distinct from solid LNP 20:0 BPD-PC (SM-102) nanoparticle controls which assume a spherical structure with an amorphous lipid aggregate core and uni/multilamellar lipid surface. LNP 20:0 BPD-PC (without SM-102) were unstable and were therefore not imaged using cryo-TEM. As mentioned before, unlike liposomes, solid LNPs are not amenable to hydrophilic agent encapsulation.

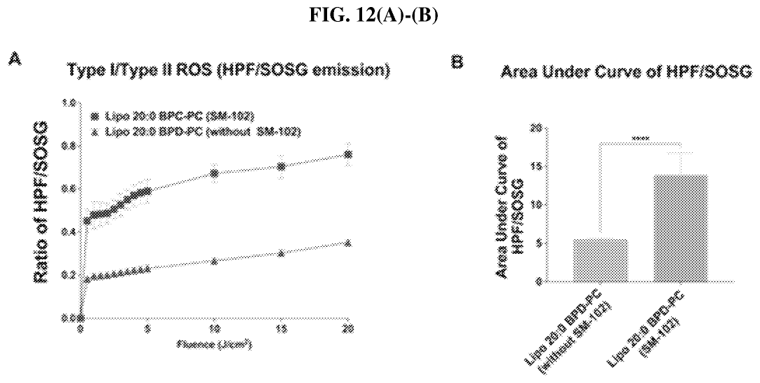

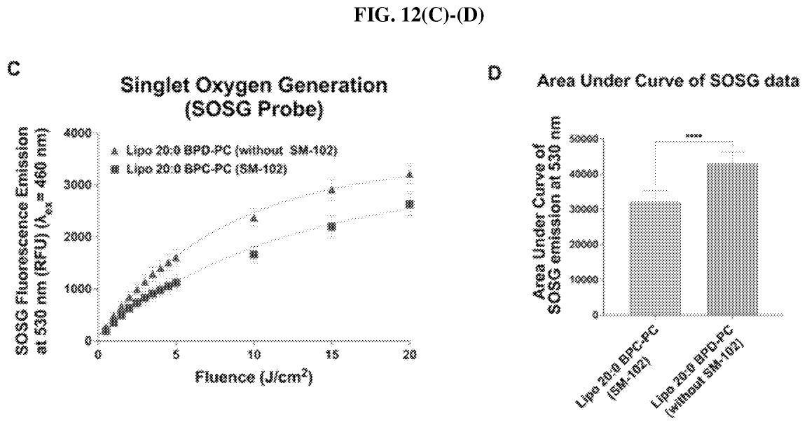

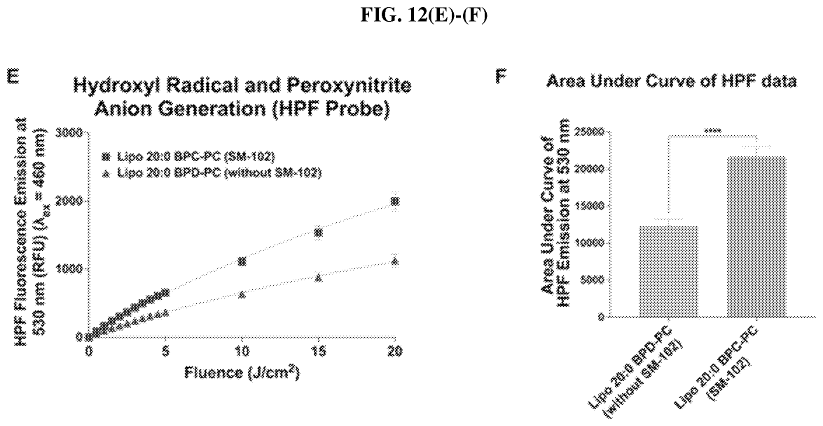

Quantifying ROS production: Upon light activation, photosensitizers undergo Type I and Type II photochemical reactions that contribute to their therapeutic action during PDT and photodynamic priming. Type II mechanisms generate singlet oxygen through energy transfer from the excited triplet state photosensitizer to molecular oxygen, while Type I mechanisms lead to the generation of radicals, such as hydroxyl radical, through electron transfer processes. As we have recently discovered in our mechanistic study on light activated lipid nanoformulations, a shift from Type I to Type II ROS is conducive to increased efficiencies in photochemical ICD in cancer cells. In this study, both Type I and Type II ROS were quantified using fluorescent probes: SOSG for singlet oxygen and HPF for hydroxyl radical and/or peroxynitrite anion. These probes exhibit an increase in fluorescence emission when they interact with specific ROS molecules. FIG. 12 shows generation of reactive oxygen species (ROS) by light-activated liposomes containing BPD-PC measured using fluorescent probes, including (A) the ratio of HPF/SOSG and (B) the area under the curve of HPF/SOSG, (C and D) Singlet oxygen production measured using the fluorescent probe Singlet Oxygen Sensor Green (SOSG), l_{Exc}=460 nm and l_{Emi}=530 nm, and (E and F) Hydroxyl radical and/or peroxynitrite anion generation measured using the fluorescent probe hydroxyphenyl fluorescein (HPF), l_{Exc}=460 nm and l_{Emi}=530 nm. (All the data is presented as mean±S.D., (n=12), statistical significance was calculated using a one-way ANOVA test using GraphPad Prism v10.4.1, *: P<0.1, **: P<0.01, ***: P<0.001, ****: P<0.0001).

As shown in FIG. 12(A)-(B), incorporation of SM-102 into the liposomes led to 2.5-fold elevations in Type I/Type II ROS ratios (HPF/SOSG). FIG. 12(C-F) demonstrate that SM-102 inclusion in liposomes (Lipo 20:0 BPD-PC (with SM-102)) enhances hydroxyl radical and/or peroxynitrite anion generation by 1.8-fold and lowers singlet oxygen production by 1.4-fold. SM-102 inclusion in 20:0 BPD-PC liposomes is therefore anticipated to improve the efficiency of photochemical ICD in cancer cells because it increases the Type I/Type II ROS ratio. This inverse relationship between Type I and Type II ROS suggests that modifications, such as the inclusion of the ionizable lipid SM-102, may shift the photochemical balance towards radical-based pathways. The increase in Type I/Type II ROS ratio that is observed with SM-102 incorporation aligns with prior reports that incorporation of electron-rich lipids or molecular components can bias photosensitizers towards Type I pathways, enhancing radical-based ROS generation. Conjugation of photosensitizers to the electron-donating molecule biotin shifts photochemical reactions toward Type I pathways to enhance superoxide anion (O₂-.) generation. Similarly, encapsulating mTHPP in electron-rich PDPA micelles shifts photochemical reactions towards Type I pathways to also enhance O₂-. generation, as compared to encapsulation in PEG-b-PLA micelles.

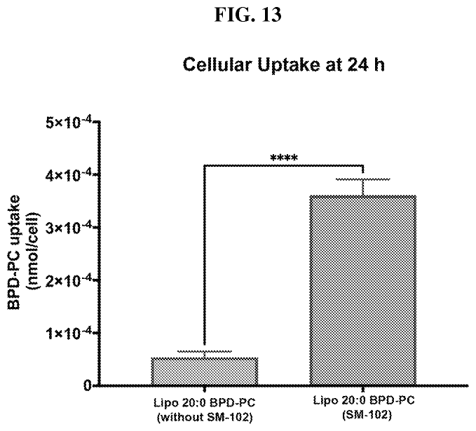

Cellular uptake: Related to the mechanisms of photochemical ICD induced by light activated lipid nanoformulations, the efficiency of cellular uptake has been identified as a key attribute that is associated with ICD marker exposure. The uptake of 20:0 BPD-PC in CT1BA5 cells delivered by Lipo 20:0 BPD-PC (SM-102) and Lipo 20:0 BPD-PC (without SM-102) has been quantified. SM-102 incorporation into liposomes increases the efficiency of 20:0 BPD-PC uptake by 6.9-fold to 3.60×10⁻⁴ nanomoles of BPD-PC equivalent per cell. FIG. 13 shows CT1BA5 cellular uptake of 20:0 BPD-PC delivered by Lipo 20:0 BPD-PC (without SM-102), Lipo 20:0 BPD-PC (SM-102), and LNP 20:0 BPD-PC (SM-102) at 24 h. (All the data is presented as mean=S.D., (n=3), statistical significance was calculated using a one-way ANOVA test on GraphPad Prism v10.4.1, *: P<0.1, **: P<0.01, ***: P<0.001, ****: P<0.0001).

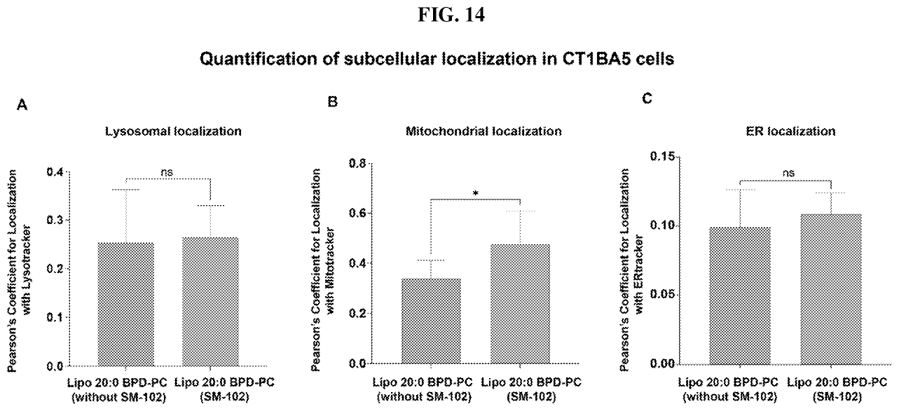

Determining Subcellular Localization: The subcellular distribution of photosensitizers plays an important role in determining the efficacy of PDT and can influence the type of cell death induced, as damage to specific organelles may trigger distinct cellular responses. In particular, direct photodamage to the endoplasmic reticulum (ER) has been reported to be a key factor in regulating photochemical ICD. For example, hypericin, a photosensitizer that preferentially localizes to the ER, was among the first agents extensively studied for its ability to induce ICD following PDT. However, several other photosensitizers capable of inducing photochemical ICD have also been reported to localize primarily to lysosomes, mitochondria, or multiple intracellular compartments simultaneously. Verteporfin-based lipid nanoparticles (V-LNPs) exhibiting increased localization within mitochondria and the ER tend to generate stronger ICD-associated responses, including enhanced expression of ICD markers. These findings highlight the importance of organelle targeting in shaping PDT-induced immune responses. To further investigate the impact of SM-102 incorporation on intracellular distribution, the subcellular localization of the liposomal formulations was quantified in CT1BA5 cells by calculating Pearson's correlation coefficients for co-localization with specific organelle markers using confocal microscopy. FIG. 14 shows Pearson's coefficient values for colocalization of Lipo 20:0 BPD-PC (with SM-102) and Lipo 20:0 BPD-PC (without SM-102) with markers for (A) Lysosomes, (B) Mitochondria, and (C) Endoplasmic reticulum (ER) in CT1BA5 cells. (All data are presented as mean #S.D., (n=6). Statistical significance was calculated using a one-way ANOVA test on GraphPad Prism v10.4.1, *: P<0.1, **: P<0.01, ***: P<0.001, ****: P<0.0001).

Both Lipo 20:0 BPD-PC (without SM-102) and Lipo 20:0 BPD-PC (with SM-102) exhibited localization within lysosomes. However, Lipo 20:0 BPD-PC (with SM-102) also demonstrated significantly greater localization of the photosensitizer within mitochondria compared with Lipo 20:0 BPD-PC (without SM-102), which, according to PCA, is conducive to increased photochemical ICD. This is likely because the clinical ionizable lipid SM-102 acts to stabilize mRNA sequences within clinical LNPs and assists with endosomal escape upon acidification of the endo-lyosomes. Within clinical LNPs, mRNA escape allows for mRNA translation, while for these liposomes, it appears that SM-102 facilitates partial translocation of the photosensitizer to the mitochondria to augment photochemical ICD.

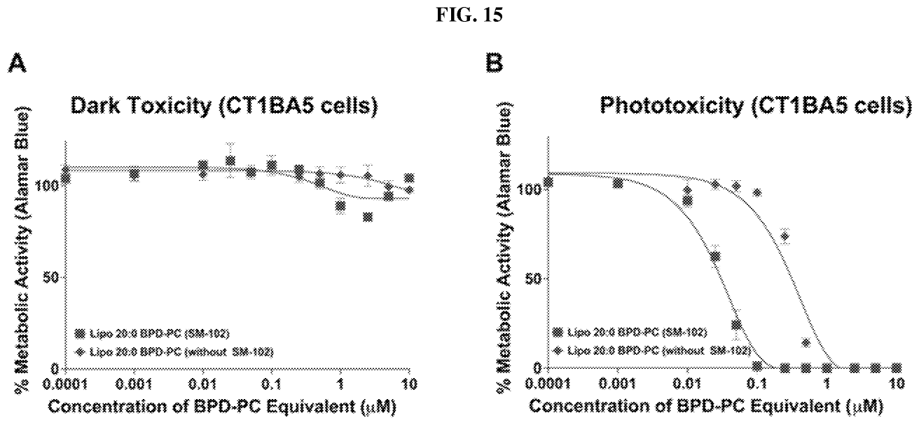

Measuring phototoxicity: The impact of PDT using Lipo 20:0 BPD-PC (SM-102) and Lipo 20:0 BPD-PC (without SM-102) with and without light exposure on the metabolic activity of CT1BA5 cells was examined. FIG. 15 shows metabolic activity of CT1BA5 cells as determined by the Alamar Blue assay following (A) no PDT and (B) PDT using all the formulations activated by 690 nm light with a fluence of 20 J/cm² (All the data is presented as mean±S.D., (n=3), statistical significance was calculated using a one-way ANOVA test using GraphPad Prism v10.4.1, *: P<0.1, **: P<0.01, ***: P<0.001, ****: P<0.0001). As shown in FIG. 15(A), incorporation of SM-102 did not impact the dark toxicity of the liposomes as neither formulations exhibited any noteworthy reductions in metabolic activity with up to 10 μM BPD-PC equivalent of liposomes in the absence of light. Upon light activation (FIG. 15B), Lipo 20:0 BPD-PC (SM-102) resulted in a greater decrease in metabolic activity with an IC₅₀ of 30.4 nM, while Lipo 20:0 BPD-PC (without SM-102) was 10-fold less phototoxic. The IC₂₅, IC₅₀, and IC₇₅ values for each formulation were extrapolated from the cell viability curves obtained using an Alamar Blue assay. The trends in phototoxicity levels were consistent with the trends in increased cellular uptake with SM-102 incorporation.

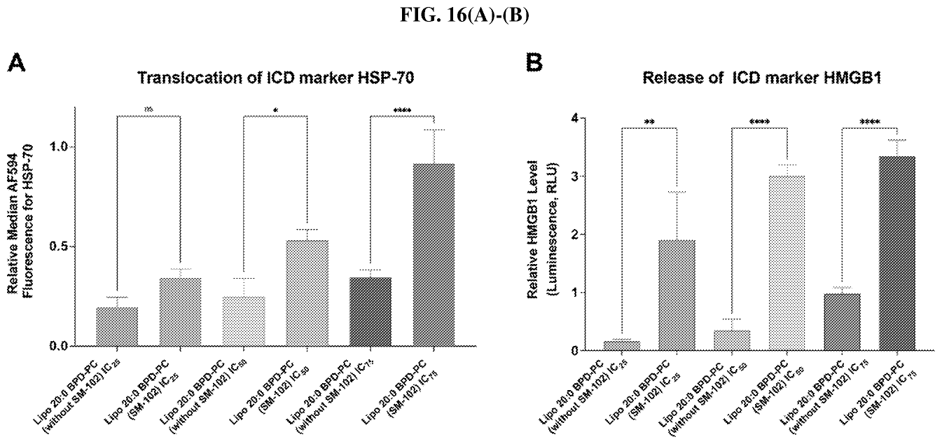

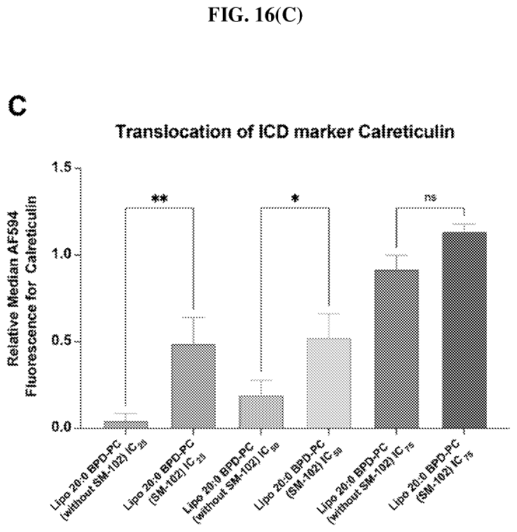

Measuring photochemical immunogenic cell death: PDT can induce ICD through the release or translocation of DAMPs, such as HSP-70, calreticulin, and HMGB1. Both in vitro and in vivo that liposomal BPD formulations effectively induce ICD in pancreatic and head and neck cancers. Light activated solid LNPs containing SM-102, which are modeled on the SPIKEVAX vaccine, are more efficient at inducing photochemical ICD than conventional BPD-PC liposomes. The impact of incorporating SM-102 into liposomes containing BPD-PC on the induction of photochemical ICD under three conditions (IC₂₅, IC₅₀, and IC₇₅) was investigated by measuring the exposure of HSP-70, calreticulin, and HMGB1 following PDT in CT1BA5 cells. FIG. 16 shows exposure of the ICD marker HSP-70 in CT1BA5 cells post-PDT using 690 nm light with a fluence of 20 J/cm² and IC₂₅, IC₅₀, and IC₇₅ concentrations of Lipo 20:0 BPD-PC (SM-102) and Lipo 20:0 BPD-PC (without SM-102). (All the data was normalized to untreated cells and is presented as mean±S.D., (n=3). Statistical significance was calculated using a one-way ANOVA test on GraphPad Prism v10.4.1, *: P<0.1, **: P<0.01, ***: P<0.001, ****: P<0.0001) The inclusion of SM-102 into liposomes increases the efficiency of HSP-70, calreticulin, and HMGB1 exposure following light activation at all three inhibitory concentrations, and HSP-70, calreticulin, and HMGB1 exposure was proportional to the PDT dose used. Interestingly, incorporating SM-102 into liposomes enables an even more efficient exposure of HSP-70 exposure following light activation at the IC₇₅ BPD-PC equivalent concentration, showing a 1.5-fold increase compared to the LNP 20:0 BPD-PC (SM-102) formulation. This could be attributed to the fact that Lipo 20:0 BPD-PC (SM-102) has a higher ratio of Type I/Type II ROS than the LNP 20:0 BPD-PC (SM-102) control formulation, which is an important feature of light activated lipid nanoformulations for efficient photochemical ICD marker exposure.

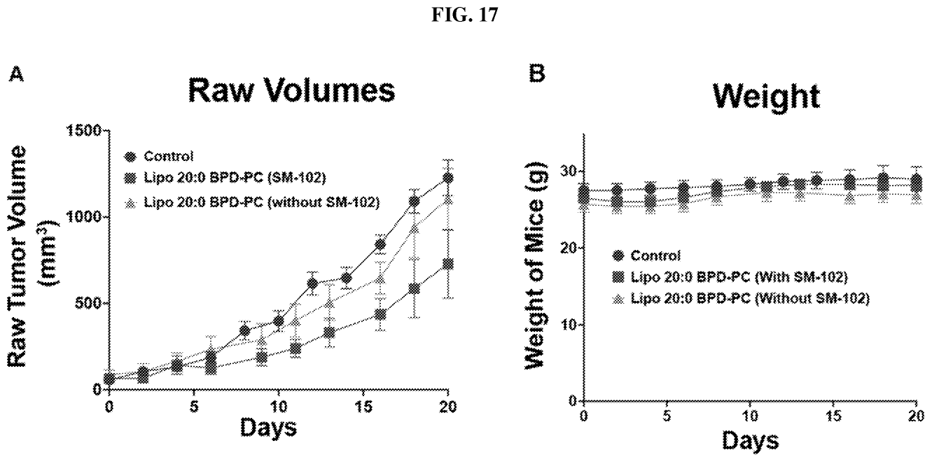

PDT using Lipo 20:0 BPD-PC (with SM-102) improves tumor control in an immune-cold PDAC model: To evaluate whether formulation-dependent differences in photochemistry and ICD induction in vitro translate to therapeutic efficacy in vivo, Lipo 20:0 BPD-PC (with SM-102) and Lipo 20:0 BPD-PC (without SM-102) with PDT were compared in an immune cold murine PDAC model. CT1BA5 pancreatic tumors were established in C57BL/6 mice and treated with a single intravenous dose of 0.58 mg/kg BPD-equivalent dose for both the formulations followed by irradiation with 690 nm light. FIG. 17 shows (A) Tumor volumes of mice until day 20, and (B) Body weights, which remained stable across groups, indicating no systemic toxicity associated with PDT treatment. (Data are mean±S.E.M.; statistical significance was calculated using one-way ANOVA with a Tukey post-test on GraphPad Prism v11.0.0; statistical significance for survival data was calculated using Log-rank (Mantel-cox test) on GraphPad Prism v11.0.0; *p<0.05, **p<0.01, ***p<0.001). As shown in FIG. 17, both formulations delayed tumor growth relative to untreated controls. However, PDT with Lipo 20:0 BPD-PC (with SM-102) resulted in greater tumor growth inhibition compared with Lipo 20:0 BPD-PC (without SM-102). Tumor volume measurements demonstrated improved tumor control in mice receiving Lipo 20:0 BPD-PC (with SM-102), whereas tumors treated with Lipo 20:0 BPD-PC (without SM-102) exhibited comparatively faster progression. Body weights remained stable across all treatment groups, indicating that PDT treatment was well tolerated and did not produce significant systemic toxicity. These findings suggest that incorporation of SM-102 within the liposomal formulation enhances the therapeutic efficacy of PDT in immune-cold PDAC tumors, potentially through improved photochemical ICD of cancer cells.

This example demonstrates that the incorporation of the clinical ionizable lipid SM-102 into verteporfin-conjugated liposomes significantly enhances photochemical ICD by increasing the production of Type I ROS (hydroxyl radical and/or peroxynitrite anion), the efficiency of cellular uptake, and relocating of the photosensitizer to the mitochondria. High Type I ROS production, high efficiency of cellular uptake, and high localization in the mitochondria are among several key attributes of verteporfin-lipid nanoformulations that are strongly associated with the photochemical induction of ICD marker exposure. Incorporation of SM-102 influenced the intracellular trafficking of these liposomes, resulting in increased mitochondrial localization compared with conventional verteporfin-lipid conjugate liposomes. Since mitochondrial and endoplasmic reticulum photodamage have been associated with stronger ICD signaling, these localization differences may further contribute to the enhanced immune-stimulatory responses observed with SM-102-containing liposomes. The incorporation of the clinical ionizable lipid SM-102 is a simple and clinically viable method of improving these key attributes of verteporfin-lipid conjugate liposomes without negatively impacting their dark toxicity profile in vitro or systemic toxicity in vivo. As such, SM-102 doped verteporfin-lipid conjugate liposomes appear to be an attractive candidate for in vivo photochemical immune stimulation. Consistent with these in vitro findings, preliminary in vivo studies in an immune-cold PDAC model demonstrated that SM-102 containing liposomes achieved improved tumor growth inhibition following PDT compared with conventional verteporfin-lipid conjugate liposomes, supporting the translational potential of this formulation strategy. These liposomes can also capitalize on the option for multi-agent co-encapsulation with complimentary therapeutic agents, such as chemotherapy, to move towards a combination regimen with greater potential for immune-enhancing PDT.

Integrating SM-102 into light-activated liposomes provides a simple and robust method of improving the efficiency of photochemical ICD and holds potential for enhancing immunotherapy-based combinations that benefit from liposomal drug delivery systems.

### Example 3

Pancreatic ductal adenocarcinoma (PDAC) is among the most aggressive and deadly cancer, marked by a dense desmoplastic stroma, extensive immune suppression, and poor responsiveness to current therapeutic interventions. For patients with unresectable tumors, fluorouracil- or gemcitabine-based chemotherapy regimens, administered with or without radiation therapy, continue to represent the standard of care. Although incremental benefits have been achieved with chemotherapy, PDAC continues to be associated with dismal clinical outcomes, underscoring the necessity for novel therapeutic approaches.

Immune checkpoint blockade (ICB) has transformed the treatment landscape for several malignancies, including melanoma, non-small cell lung cancer, urothelial carcinoma, and triple-negative breast cancer. In particular, antibodies targeting programmed death ligand-1 (α-PD-L1) restore cytotoxic T-cell activity by disrupting inhibitory signaling between tumor cells and immune cells. However, the clinical efficacy of α-PD-L1 therapy in PDAC has been limited. This resistance is largely attributed to the immune-cold, non-T-cell-inflamed tumor microenvironment and inadequate endogenous priming of antitumor immunity. Consequently, PDAC has shown minimal responsiveness to ICB monotherapy, with clinical trials reporting overall response rates of approximately 0% for single-agent blockade and only ~3% for combined α-PD-1 and a-CTLA-4 therapy. These findings suggest that PD-1/PD-L1 inhibition alone is insufficient and that additional combination strategies capable of enhancing immune activation and tumor priming are required to improve therapeutic outcomes in PDAC.

Photodynamic therapy (PDT) is a non- to minimally invasive treatment modality that enables spatially and temporally controlled tumor killing through light activation. In addition to inducing direct cytotoxicity in tumor cells and damaging the tumor vasculature, PDT has demonstrated significant preclinical potential to stimulate antitumor immune responses. PDT has been shown to increase immunogenicity in tumors by inducing immunogenic cell death (ICD), a distinct mode of cell death that stimulates both innate and adaptive immune responses. Stressed or dying cell releases or translocate pro-inflammatory cytokines, damage-associated molecular patterns (DAMPs), and tumor-associated antigens (TAAs). These factors collectively facilitate antigen uptake, processing and presentation by immune cells, ultimately promoting tumor-specific immune responses. Key DAMPs associated with ICD include surface exposure of calreticulin (CRT), release of high mobility group box 1 (HMGB1) and heat shock proteins (HSPs). Through these mechanisms, ICD can shift the tumor microenvironment toward an immune-stimulatory state, promoting T-cell activation, proliferation, and infiltration, thereby enhancing the efficacy of existing immunotherapies.

Integrating PDT with ICB can potentiate antitumor immune responses. A nanoplatform that simultaneously delivers α-PD-L1 antibodies and the photosensitizer chlorin e6 (Ce6) has been designed, enabling coordinated light-activated immune stimulation together with checkpoint inhibition in melanoma models. In another approach, photochemical internalization has been applied to promote light-dependent intracellular release of an α-PD-L1 immunotoxin, which increased cytotoxic activity in breast cancer systems and induced regression of both treated lesions and distant tumors through systemic immunity. The use of redaporfin, a clinically advancing photosensitizer, combined with α-CTLA-4 blockade have shown strong therapeutic responses in syngeneic colorectal and breast tumor models, including improved tumor control and reduced metastatic. Photodynamic priming (PDP) using Visudyne® enhances the therapeutic efficacy of nanoliposomal irinotecan while simultaneously converting immune-silent pancreatic tumors into immune-responsive ones. Mechanistically, PDP increased intratumoral drug accumulation, promoted immune effector cell infiltration, and amplified ICD. These changes collectively improved responsiveness to anti-PD-1 therapy, suggesting that PDP may broaden the effectiveness of immune checkpoint inhibitors in PDAC. However, one of the limitations of Visudyne®-based formulations is the relatively limited stability of BPD within liposomal carriers, which can result in premature drug leakage. To address this limitation, lipid-BPD conjugates as described herein were engineered that enable stable insertion of the photosensitizer within lipid bilayers or the lipid nanoparticle core, thereby improving formulation stability and reducing premature drug leakage from lipid based carriers. PD-L1-targeted photoactivatable liposomes (iTPALs) enhance penetration and distribution within the dense stromal environment of PDAC. This system enables self-delivery of the 20:0 BPD-PC photosensitizer while enhancing checkpoint inhibition, likely through multivalent engagement mediated by surface-conjugated α-PD-L1 antibodies. Furthermore, a mechanistic framework linking verteporfin-lipid nanoparticles (V-LNPs) design to photochemical ICD, identifying Type I ROS generation, intracellular localization, and phototoxicity as key determinants of immunogenic outcomes in vitro has been developed. These findings allow for nanoparticle engineering to modulate tumor immune contexture and overcome resistance to checkpoint blockade.

This example directly compares verteporfin-based nanoplatforms, LNP BPD-Cholesterol and Lipo 20:0 BPD-PC, representing the most and least efficient photochemical ICD inducers, to demonstrate how differences in ROS-driven photochemistry translate into distinct molecular and cellular responses. An immune-cold, non-T-cell-inflamed PDAC model was employed to determine whether photochemically tuned ICD can convert poorly responsive cold tumors into responsive hot tumors. Using bulk transcriptomic profiling, pathways associated with immune activation, and enhanced susceptibility to α-PD-L1 therapy, where combination of LNP BPD-Cholesterol PDT and α-PD-L1 therapy improves survivability compared with monotherapy of LNP BPD-Cholesterol PDT or α-PD-L1 therapy. Collectively, this example demonstrates a translational strategy to enhance immunotherapy outcomes in immune cold pancreatic cancer.

### Material and Method

Conjugation of BPD with 20:0 Lyso PC

To prepare the verteporfin lipid conjugate (20:0 BPD-PC), BPD was conjugated to 1 arachidoyl-2-hydroxy-sn-glycero-3-phosphatidylcholine (20:0 lyso PC). Briefly, 20:0 lyso PC, BPD (US Pharmacopeia), 1-ethyl-3-(3-dimethylaminopropyl) carbodiimide (EDC; Sigma-Aldrich), 4-(dimethylamino) pyridine (DMAP; Sigma-Aldrich), and N, N-diisopropylethylamine (DIPEA; Sigma-Aldrich) were combined in a molar ratio of 1:5:50:25:60, respectively, in 5 mL of ethyl acetate. After the reaction mixture was briefly vortexed, it was stirred at 2500 rpm for 72 h at room temperature. The solvent was removed from the reaction mixture using nitrogen gas and the resulting dried reaction mixture residue was redissolved in 2 mL of a mobile phase that contained chloroform, methanol and water combined in a 65:25:1 ratio. A chromatography column was packed with a silica gel slurry consisting of Silica Gel 60 (Sigma-Aldrich) and 10 mL of the mobile phase. After the chromatography column was conditioned with 10 mL of the mobile phase, the redissolved reaction mixture was transferred into the column and allowed to fully enter the stationary phase, before elution with an additional 15 mL of the mobile phase. The slowest fraction containing the conjugated product (20:0 BPD-PC) was collected and concentrated by rotary evaporation at a temperature of 30° C. After being redissolved in 1 mL of chloroform and filtered through a 0.22 μm syringe filter (VWR), the purified 20:0 BPD-PC residue was redried by rotatory evaporation at 30° C. and redissolved in 1 mL of chloroform. An additional four chloroform washes (total of 5 mL) were performed to collect the remaining 20:0 BPD-PC. The final purified 20:0 BPD-PC product was stored in a glass vial at −20° C. in the dark.

Conjugation of BPD with Cholesterol

To prepare the verteporfin lipid conjugate (BPD-Cholesterol), BPD was conjugated to cholesterol. In short, cholesterol, BPD (US Pharmacopeia), 1-ethyl-3-(3-dimethylaminopropyl) carbodiimide (EDC; Sigma-Aldrich), 4-(dimethylamino) pyridine (DMAP; Sigma-Aldrich), and N, N-diisopropylethylamine (DIPEA; Sigma-Aldrich) were combined in a molar ratio of 1:5:50:25:60, respectively, in 5 mL of ethyl acetate. After briefly vortexing the reaction mixture, it was stirred at 2500 rpm for 72 h at room temperature. The mixture was transferred to a chromatography column that was packed with a silica gel slurry consisting of Silica Gel 60 (Sigma-Aldrich) and 10 mL of an ethyl acetate (mobile phase). This was followed by elution with an additional 15 mL of the mobile phase. The fastest fraction containing the eluate of the conjugated product (BPD-Cholesterol) was collected. The ethyl acetate in the resulting sample was evaporated using nitrogen gas and the dried film was reconstituted in 1 mL of a solvent mixture containing ethyl acetate and n-Hexanes combined in a 1:1 ratio. The reconstituted mixture was run through the chromatography column a second time. The fastest fraction containing the conjugated product (BPD-Cholesterol) was collected and concentrated by rotary evaporation at a temperature of 30° C. The dried BPD-Cholesterol residue was redissolved in 1 mL of chloroform. An additional four chloroform washes (total of 5 mL) were performed to collect the remaining BPD-Cholesterol. The final purified BPD-Cholesterol product was stored in a glass vial at −20° C. in the dark.

Synthesis of Liposome with 20:0 BPD-PC

To synthesize Liposomal (Lipo) 20:0 BPD-PC, 1,2-Dipalmitoyl-sn-glycero-3-phosphatidylcholine (DPPC, Avanti), 1,2-Dimyristoyl-sn-glycero-3-phosphatidylcholine (DMG-PEG, NOF America Corporation), cholesterol, and 20:0 BPD-PC were combined at a molar ratio of 0.590:0.015:0.385:0.010, respectively, in a 5 mL glass vial. The molar ratio of lipids for Lipo 20:0 BPD-DC without SM-102 was determined based on our previously published formulations. The liposome was prepared using the conventional thin-film hydration method. After evaporating the chloroform from the lipid mixture using nitrogen gas, the resulting dried lipid film was hydrated with 1 mL of DPBS. The hydrated lipid film was heated in a water bath at 42° C. for 30 min, vortexed for 10 min, and subjected to ultrasonication (Ultrasonic Probe Sonicator; Fisher Scientific) for a total of 30 min (20 s on/40 s off cycles) at 42° C. in the dark. The resulting Lipo 20:0 BPD-PC without SM-102 formulation was stored at 4° C. in the dark.

Synthesis of LNP with BPD-Cholesterol

To synthesize the solid lipid nanoparticle (LNP) with BPD-Cholesterol, 2-Dipalmitoyl-sn-glycero-3-phosphatidylcholine (DPPC, Avanti), cholesterol, 1,2-Dimyristoyl-sn-glycero-3-phosphatidylcholine (DMG-PEG, NOF America Corporation), BPD-Cholesterol, and the ionizable lipid 1-octylnonyl 8-{(2-hydroxyethyl)[6-oxo-6(undecyloxy)hexyl]amino}octanoate (SM-102); BroadPharm) were combined at a molar ratio of 0.090:0.385:0.015:0.010:0.500, respectively, in a 5 mL glass vial. The molar ratio of lipids for LNP BPD-Cholesterol was determined. After evaporating the chloroform from the lipid mixture using nitrogen gas, 0.25 mL of ethanol was added to the resulting dried lipid film. The lipid solution in ethanol was vortexed for 10 min at 2000 rpm and then added dropwise into 0.75 mL of a citrate buffer solution (pH 4.0) under continuous stirring on a magnetic stir plate at 2500 rpm using a syringe pump flowing at a rate of 0.8 mL/min. The sample was left at room temperature to stir continuously for 18 h to form the LNP BPD-Cholesterol formulation. The resulting LNP mixture was dialyzed using a 100 kDA molecular weight cut-off dialysis membrane (Spectrum Labs) that was placed in a beaker containing 1 L of 1×DPBS prepared by diluting 100 mL of 10×DPBS with 900 mL of Milli-Q water and stirred at 150 rpm. The DPBS solution was replaced after every 8 h over the course of 24 h. The resulting LNP BPD-Cholesterol formulation was stored at 4° C. in the dark for future use.

### Tumor Implantation, PDT, Survival and RNA Sequencing

All procedures were performed in accordance with Institutional Animal Care and Use Committee (IACUC) protocols (Protocol number: 19-10). Male C57BL/6 mice (20 g, 6 weeks old) purchased from Jackson Laboratory were maintained in a fully equipped animal facility in compliance with the NIH Guide for the Care and Use of Laboratory Animals at the University of Texas at Dallas Vivarium. A day prior to implantation, the hair on the left flank of mice were shaved with Wahl professional animal hair clippers. CT1BA5 cells were cultured in DMEM containing 10% FBS. When the cells reached 80% confluency, they were trypsinized and collected in media. The cells in the media were counted and washed three times with DPBS to remove any remaining media and trypsin. The cells were then diluted to obtain 1×10⁶ cells in 20 μl sterile DPBS and implanted subcutaneously into the left flank of the mice. The tumor volumes were monitored after implantation using a digital vernier caliper. 16 days following tumor implantation, the tumors reached a volume of 50 mm³-70 mm³ as calculated using the formula ab2/2 (a=length of larger dimension, b=length of smaller dimension). On the treatment day, the mice were divided into eight cohorts: 1) Control 2) Lipo 20:0 BPD-PC 3) Lipo 20:0 BPD-PC+690 nm 4) LNP BPD-Cholesterol 5) LNP BPD-Cholesterol+690 nm 6) LNP BPD-Cholesterol+690 nm+α-PD-L1 and 7) α-PD-L1 only group with 7 mice on each arm. Before PDT, the mice were pre-emptively administered with subcutaneous Meloxicam SR analgesia to minimize any discomfort during PDT. The groups were then intravenously administered with 0.58 mg/kg of BPD-equivalent). For α-PD-L1 250 μg/mouse was injected intraperitonially every 3 day for 3 times. Immediately following administration, tumors with the PDT group were irradiated with a 690 nm laser at a fluence of 150 J/cm² and an irradiance of 100 mW/cm². Following the treatment, tumor volumes were measured in every alternate day until they die, or their volumes reach 2000 mm³ endpoint. The tumor volumes, probability of survival, area under curve (AUC) of tumor volume, progression-free survival and overall survival in mice were then plotted and analyzed using Graph Pad Prism v10.6.1. For progression-free survival analysis, 500 mm³ was used as a cutoff volume, whereas for overall survival, the day when a mouse was dead/euthanized was considered as an endpoint. Median survival was calculated automatically during overall survival analysis by Graph Pad Prism v10.6.1.

For bulk tumor RNA sequencing, mice were euthanized on day 7 post-treatment. Tumors were rapidly harvested and dissected into smaller tissue pieces, with approximately 4×4×4 mm cubes prepared for each sample. Tissue pieces were placed into 2 mL screw-cap microcentrifuge tubes, with two tubes prepared per tumor specimen. Samples were immediately submerged in RNA protect Tissue Reagent. For tumor pieces weighing approximately 40-45 mg, 450 μL of RNA protect reagent was added per tube. Following stabilization, samples were rapidly transferred to dry ice and stored at −80° C. until further processing. Frozen samples were transported on dry ice for subsequent RNA extraction and sequencing analysis.

### Differential Expression Analysis

Before the analysis of differential gene expression, the raw read counts were normalized by applying variance stabilizing transformation (VST) using DESeq2 package. Post VST, the comparative gene analysis was run using DESeq2, with p-values being adjusted by Benjamini and Hochberg method and low counts under 10 were filtered out. For inter-treatment comparisons, the log fold change was shrunk using Normal (Normal prior from Love et al (2014)) for visualizations such as heatmaps and Wald statistic from DESeq2 was used gene set enrichment analysis ranking.

### Gene Enrichment Analysis

To identify enriched biological pathways and immune contexture, fgsea package (v1.36.0) from R (Bioconductor version 3.22) was used. For each pairwise comparison, genes were pre-ranked based on the Wald statistic from DESeq2 results, accounting for both effect size and uncertainty of estimation. Immune gene set comprised of markers unique to B-cells, CD4 T-cells, CD8 T-cells, IL6-mediated systemic inflammation, IL-1 mediated systemic inflammation, natural killer cells, M1 macrophages, M2 macrophages, tumor associated macrophages (TAM), dendritic cells, neutrophils and interferon (IFN) response. Gene signature set comprised of gene markers related to desmoplasia, ferroptosis, pyroptosis, ICD markers, immunosuppressive genes and PDAC related genes. Genes with missing symbol naming or statistics as well as gene sets that had fewer than 5 genes in the list were excluded. GSEA was performed with 10,000 permutations and normalized enrichment scores (NES) and p-values were calculated. The results were visualized using dot plot graphs, plotting the pathways against NES with dot size representing number of genes and color indicating p-values.

### Results

### LNP BPD Cholesterol and Lipo 20:0 BPD-PC-Mediated PDT Improves Tumor Control in an Immune-Cold PDAC Model

Nanoparticle formulation influences the immunogenic outcomes of PDT. Systematic evaluation of formulations of V-LNPs revealed verteporfin incorporated within solid lipid nanoparticles produced higher levels of radical ROS, including hydroxyl radicals and peroxynitrite, and elicited stronger ICD signaling compared with liposomal formulations. Multivariate analysis using principal component analysis further revealed that Type I ROS generation was most strongly associated with ICD marker exposure and dendritic cell activation, whereas Type II ROS showed the weakest association. In addition, nanoparticle localization within the endoplasmic reticulum and mitochondria correlated with enhanced ICD signaling, while lysosomal localization was less strongly associated with ICD marker exposure. These findings indicated that formulation-dependent photochemistry and intracellular trafficking of lipid nanoparticles can regulate the immune-stimulatory potential of PDT.

To determine whether formulation-dependent photochemical differences translate into therapeutic efficacy in vivo, two representative formulations from the formulations of V-LNPs with different ICD-inducing capacities were selected. LNP BPD-Cholesterol, which previously exhibited strong ICD-associated responses in vitro, was compared with Lipo 20:0 BPD-PC, a formulation that produced comparatively weaker ICD signaling. Both formulations were prepared as described herein and further characterized to confirm their physicochemical properties. DLS measurements showed that LNP BPD-Cholesterol had an average hydrodynamic diameter of 147.6 nm with a polydispersity index (PDI) of 0.08, whereas Lipo 20:0 BPD-PC exhibited a smaller size of 108.3 nm with a PDI of 0.12. UV-Vis spectroscopy confirmed the characteristic verteporfin absorbance profiles for both formulations, indicating successful incorporation of the photosensitizer. Further CT1BA5 pancreatic tumors were established in male C57BL/6 mice (6 weeks old, ~20 g). When tumors reached a volume of approximately 50-70 mm³ (14 days post implantation), mice received a single intravenous administration of either LNP BPD-Cholesterol or Lipo 20:0 BPD-PC at a verteporfin dose equivalent of 0.58 mg/kg. Immediately following administration of V-LNPs, tumors were irradiated with 690 nm light at a total fluence of 150 J/cm² and an irradiance of 100 mW/cm².

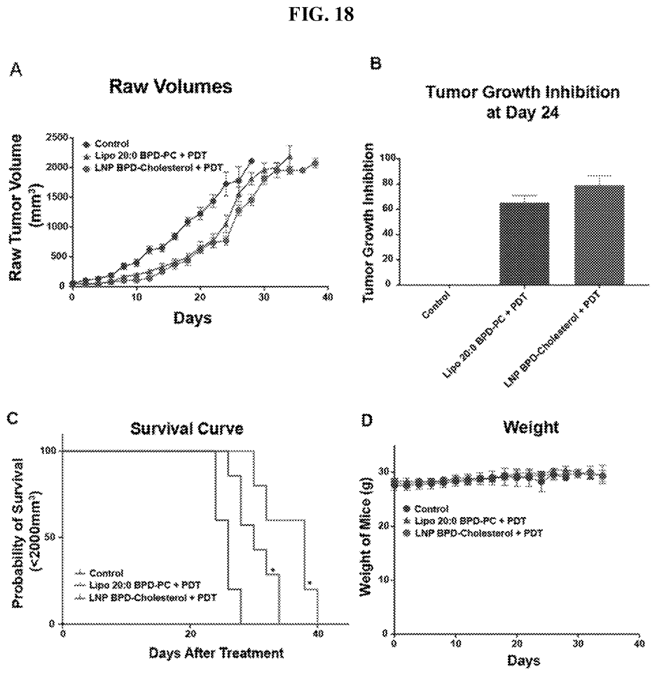

Both PDT formulations delayed tumor progression relative to untreated controls. FIG. 18 shows (A) tumor volumes of mice until day 40 when all mice died, (B) Endpoint tumor volumes measured at the experimental endpoint, (C) Kaplan-Meier plots representing the probability of overall survival in mice, and (D) Body weights, which remained stable across groups, indicating minimal toxicity associated with PDT treatment. (Data are mean±S.E.M.; statistical significance was calculated using one-way ANOVA with a Tukey post-test on GraphPad Prism v11.0.0; statistical significance for survival data was calculated using Log-rank (Mantel-cox test) on GraphPad Prism v11.0.0; *p<0.05, **p<0.01, ***p<0.001). LNP BPD-Cholesterol-mediated PDT resulted in greater tumor growth inhibition compared with liposomal PDT. Tumors treated with LNP BPD-Cholesterol exhibited more tumor growth inhibition than those treated with Lipo 20:0 BPD-PC. Kaplan-Meier survival analysis further demonstrated improved survival in mice receiving LNP BPD-Cholesterol PDT relative to Lipo 20:0 BPD-PC PDT. Body weight remained stable across treatment groups throughout the study, indicating good tolerability and minimal toxicity.

### Transcriptomic Profiling Reveals Formulation-Dependent Stress and Immune Signaling Programs

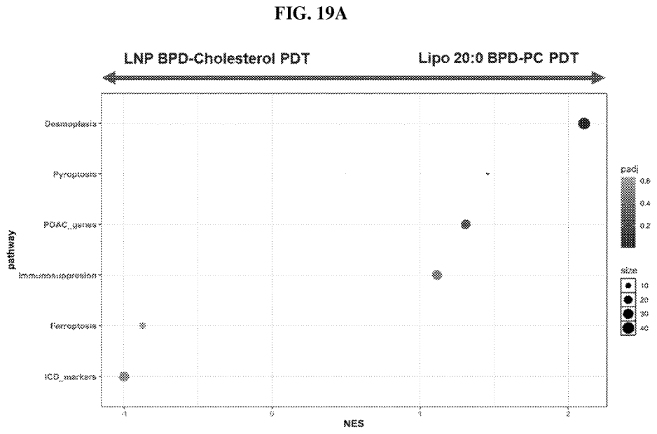

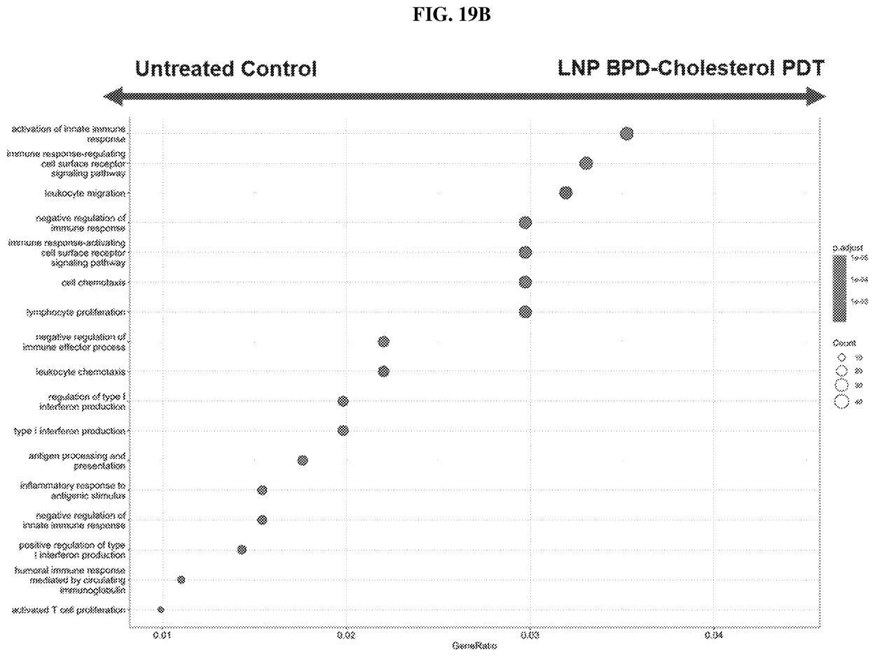

To investigate molecular differences underlying the distinct therapeutic responses, bulk RNA sequencing was performed on tumors treated with LNP BPD-Cholesterol PDT or Lipo 20:0 BPD-PC PDT. Gene set enrichment analysis (GSEA) was used to compare transcriptional programs between treatment groups. Analysis of distinct enrichment patterns revealed clear formulation-dependent differences in cell death and tumor microenvironment associated pathways. Transcriptomic profiling reveals formulation-dependent differences in immune signaling following PDT. FIG. 19A shows gene set enrichment analysis (GSEA) comparing tumors treated with LNP BPD-Cholesterol PDT relative to Lipo 20:0 BPD-PC PDT. Tumors treated with LNP BPD-Cholesterol PDT showed enrichment of ferroptosis-related pathways and ICD marker signatures, consistent with enhanced oxidative stress driven cell death. In contrast, tumors treated with Lipo 20:0 BPD-PC PDT showed stronger enrichment of gene patterns associated with desmoplasia, pyroptosis, PDAC-related transcriptional signatures, and immunosuppressive signaling, suggesting that nanoparticle formulation influences the downstream biological consequences of PDT within the tumor microenvironment.

To further assess immune modulation, immune cell-associated gene signatures were analyzed. FIG. 19B shows immune cell-associated gene signatures enriched in LNP BPD-Cholesterol PDT relative to Lipo 20:0 BPD-PC PDT. LNP BPD-Cholesterol PDT showed enrichment of CD4⁺ T-cell signatures and IL-6 associated inflammatory signaling, indicating activation of inflammatory pathways following treatment. By contrast, Lipo 20:0 BPD-PC PDT demonstrated higher enrichment of several immune cells related signatures, including macrophages, dendritic cells, NK cells, CD8⁺ T cells, and B cells, as well as interferon response pathways. These results suggest that while LNP-mediated PDT preferentially activates oxidative stress-associated cell death programs, Lipo 20:0 BPD-PC PDT is associated with broader immune cell related transcriptional signatures.

Differential gene expression analysis supported these observations. Volcano plot analysis identified multiple genes involved in immune signaling, inflammation, and oxidative stress responses that differed between treatment groups. Notably, genes including Ccl22, Ccr7, Il6 Irf7, Tap1, and Nox4 showed differential expression, consistent with changes in immune cell recruitment, antigen presentation, and redox regulation following PDT. Together, these findings demonstrate that LNP BPD-Cholesterol-mediated PDT induces distinct transcriptional programs compared with liposomal BPD-PC PDT, highlighting how the photosensitizer formulation shapes tumor stress responses and immune-related signaling pathways following treatment.

### LNP-Mediated PDT Enhances Responsiveness to PD-L1 Checkpoint Blockade

Because PDT can promote ICD and release DAMPs that stimulate antitumor immunity, the question of whether the stronger ICD signatures induced by LNP BPD-Cholesterol-mediated PDT could improve responsiveness to immune checkpoint inhibition was examined.

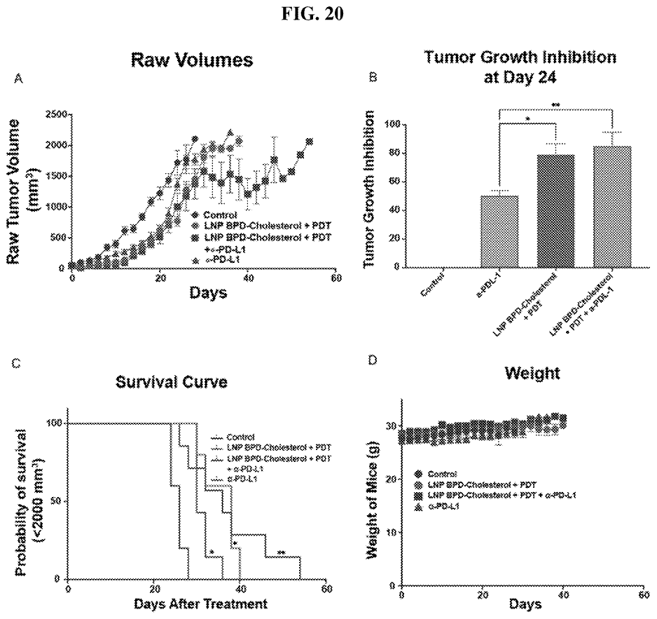

CT1BA5 tumor-bearing mice were treated with LNP BPD-Cholesterol PDT, and anti-PD-L1 antibody (250 μg per mouse) was administered intraperitoneally on days 1, 4, and 7 following PDT. Tumor growths were compared across four groups: untreated control, α-PD-L1 monotherapy, LNP BPD-Cholesterol PDT, and the combination of LNP BPD-Cholesterol PDT+α-PD-L1. FIG. 20 shows (A) Tumor volumes of mice for control, α-PD-L1, LNP BPD-Cholesterol PDT, and LNP BPD-Cholesterol PDT+α-PD-L1 treatment groups, (B) Tumors treated with the combination of LNP-mediated PDT and α-PD-L1, which exhibited tumor inhibition compared with monotherapy groups, (C) Kaplan-Meier plots representing the probability of overall survival in mice where LNP BPD-Cholesterol PDT+α-PD-L1 significantly prolonged survival relative to untreated controls and individual treatments, and (D) Body weights, which remained stable across groups, indicating minimal toxicity associated with the treatments. (Data are mean±S.E.M.; statistical significance was calculated using one-way ANOVA with a Tukey post-test on GraphPad Prism v11.0.0; statistical significance for survival data was calculated using Log-rank (Mantel-Cox test) on GraphPad Prism v11.0.0; *p<0.05, **p<0.01, ***p<0.001).

Checkpoint blockade produced minimal impact on tumor growth, consistent with the immune-cold phenotype of this PDAC model. LNP BPD-Cholesterol PDT delayed tumor progression relative to controls. Importantly, the combination of LNP PDT and α-PD-L1 produced the strongest antitumor effect, resulting in significantly delayed tumor growth compared with either monotherapy. Consistent with these observations, tumor growth inhibition was highest in the combination therapy group. Survival analysis further showed that mice receiving LNP BPD-Cholesterol PDT+α-PD-L1 exhibited the longest survival, outperforming both LNP BPD-Cholesterol PDT and α-PD-L1. Body weight remained stable throughout treatment, indicating that the combination therapy was well tolerated. Together, these results demonstrate that LNP BPD-Cholesterol PDT-mediated PDT can sensitize immune-cold pancreatic tumors to PD-L1 checkpoint blockade, resulting in improved tumor control and survival.

### PD-L1 Blockade Reshapes Immune Transcriptional Programs Following LNP-Mediated PDT

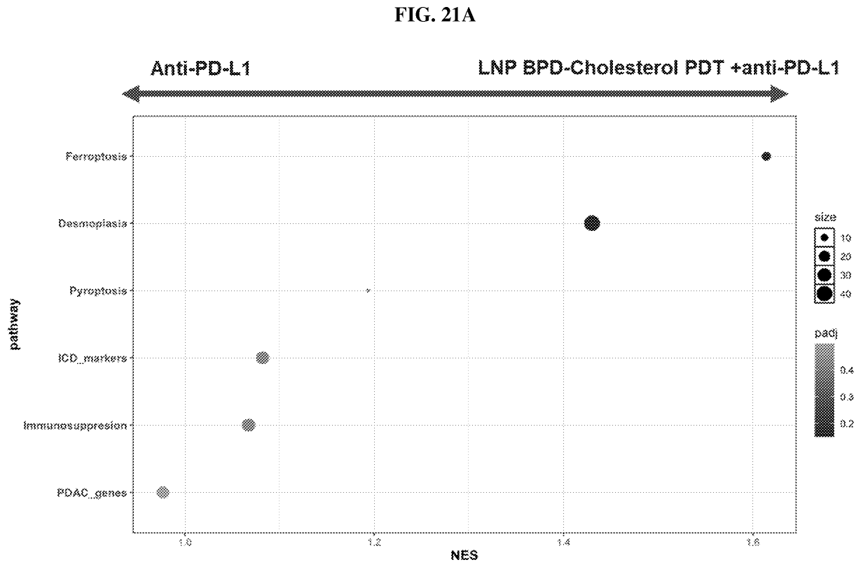

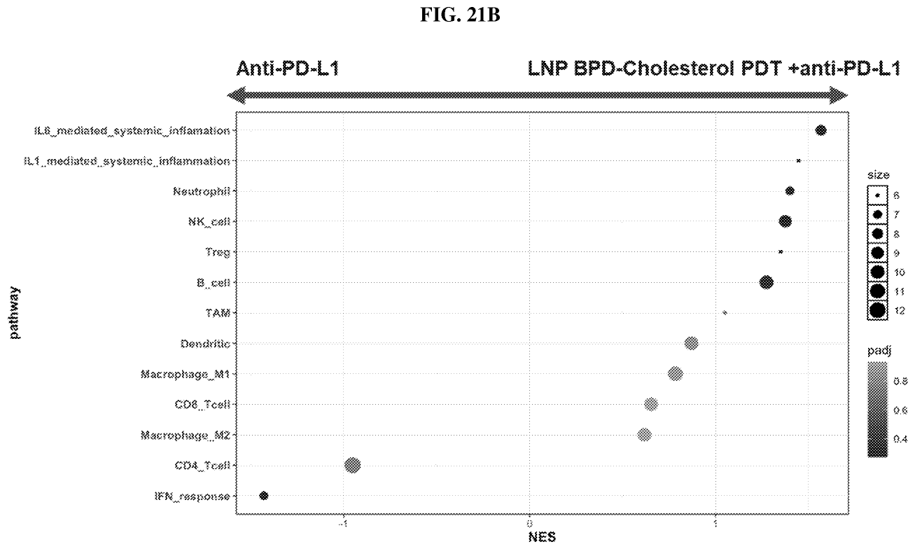

To further understand how immune checkpoint blockade alters tumor responses following PDT, transcriptomic profiles of tumors treated with LNP BPD-Cholesterol PDT+α-PD-L1 were compared with those receiving LNP BPD-Cholesterol PDT. GSEA revealed distinct enrichment patterns between the two groups. FIG. 21A shows gene set enrichment analysis (GSEA) comparing tumors treated with LNP BPD-Cholesterol PDT+α-PD-L1 relative to LNP BPD-Cholesterol PDT. Tumors treated with LNP BPD-Cholesterol PDT showed stronger enrichment of ferroptosis-related signatures, consistent with PDT-driven oxidative stress responses. In contrast, tumors treated with the combination therapy displayed enrichment of gene markers related to desmoplasia, pyroptosis, PDAC-associated gene programs, ICD marker signatures, and immunosuppressive signaling, suggesting broader remodeling of the tumor microenvironment following checkpoint blockade. FIG. 21B shows immune cell-associated gene signatures enriched in tumors treated with LNP PDT+α-PD-L1 relative to LNP BPD-Cholesterol PDT. Analysis of immune cell-associated gene signatures demonstrated that combination therapy of LNP BPD-Cholesterol PDT+α-PD-L1 increased enrichment of multiple immune cell populations, including macrophages, dendritic cells, CD4⁺ T cells, CD8⁺ cytotoxic T cells, NK cells, neutrophils, and regulatory T cells. These findings indicate that α-PD-L1 blockade enhances immune-related transcriptional patterns in tumors previously treated with PDT. Conversely, interferon response and IL-6 associated inflammatory signaling were relatively enriched in tumors treated with LNP BPD-Cholesterol PDT.

Differential gene expression analysis depicted with volcano plots further highlighted genes associated with immune activation and antigen presentation following combination therapy. Upregulated genes included Cd86, B2m, Cd8a, Xcr1, and P2rx7, which are linked to antigen presentation, dendritic cell activation, and cytotoxic T-cell responses. In contrast, stress response genes such as Atf4 and Hspa1 family members were relatively reduced following combination therapy, suggesting alterations in tumor stress adaptation pathways. Collectively, these transcriptomic analyses indicate that LNP BPD-Cholesterol-mediated PDT primarily induces oxidative stress-associated tumor cell death pathways, while the addition of PD-L1 blockade promotes broader immune activation within the tumor microenvironment.

This example demonstrates that V-LNPs with enhanced ICD-inducing capacity can significantly reshape the antitumor immune responses generated following PDT in vivo. Comparing two representative formulations with distinct photochemical properties shows that LNP BPD-Cholesterol generates stronger antitumor responses in vivo compared with Lipo 20:0 BPD-PC. Tumors treated with LNP BPD-Cholesterol PDT exhibited delayed tumor progression and prolonged survival, compared to Lipo 20:0 BPD-PC PDT. Transcriptomic profiling further revealed that LNP BPD-Cholesterol mediated PDT induces distinct molecular programs associated with oxidative stress-driven cell death, including enrichment of ferroptosis and ICD signatures. In contrast, Lipo 20:0 BPD-PC PDT was associated with transcriptional pathways linked to immunosuppressive signaling. These findings highlight how nanoparticle formulation can influence the biological pathways activated within the tumor microenvironment following PDT.

Importantly, LNP BPD-Cholesterol mediated PDT enhances responsiveness to immune checkpoint blockade. While α-PD-L1 monotherapy produced minimal therapeutic benefit in this immune-cold PDAC model, combination therapy with LNP BPD-Cholesterol mediated PDT significantly improved tumor control and survival. Transcriptomic analysis following combination treatment further indicated increased immune cell associated gene signatures and enhanced antigen presentation pathways, suggesting that PDT-induced immune priming contributes to improved checkpoint inhibitor responsiveness.

Together, these findings establish that LNP BPD Cholesterol PDT can modulate tumor microenvironment and reshape tumor-immune interactions following PDT and provide a framework for the rational design of lipid nanoparticle photosensitizers that enhance immunogenic tumor cell death and improve the efficacy of immunotherapy in immune cold cancers such as PDAC.

What is claimed is:

1. Photoactivatable nanoparticles for use as therapeutic agents in immunotherapy treatments, comprising:
lipid nanoparticles, wherein the lipid nanoparticles are liposomes or solid lipid nanoparticles, and wherein the lipid nanoparticles comprise phospholipids and lipids bound to polyethylene glycol; and
photosensitizers incorporated within the nanoparticles, wherein the photosensitizers are benzoporphyrin derivative or chemical variants of benzoporphyrin derivative, and wherein the photoactivatable nanoparticles generate reactive oxygen species through light activation of the photosensitizer.

2. The photoactivatable nanoparticles of claim 1, wherein the benzoporphyrin derivative is bound to phosphatidylcholine or cholesterol.

3. The photoactivatable nanoparticles of claim 2, wherein the benzoporphyrin derivative is bound to 20:0 lysophosphatidylcholine or 16:0 lysophosphatidylcholine.

4. The photoactivatable nanoparticles of claim 1, wherein the lipid nanoparticles further comprise ionizable lipids.

5. The photoactivatable nanoparticles of claim 4, wherein the ionizable lipids comprise SM-102.

6. The photoactivatable nanoparticles of claim 1, wherein the photoactivatable nanoparticles generate Type I reactive oxygen species.

7. The photoactivatable nanoparticles of claim 1, wherein the photoactivatable nanoparticles induce immunogenic cell death.

8. The photoactivatable nanoparticles of claim 1, wherein the photoactivatable nanoparticles localize to endoplasmic reticulum and mitochondria of the cancer cells.

9. A method for immunotherapy in a subject, comprising:
delivering photoactivatable nanoparticles to cancer cells of the subject for use as therapeutic agents, wherein the photoactivatable nanoparticles comprise lipid nanoparticles and photosensitizers incorporated within the lipid nanoparticles, wherein the lipid nanoparticles are liposomes or solid lipid nanoparticles, wherein the lipid nanoparticles comprise phospholipids and lipids bound to polyethylene glycol, wherein the photosensitizers are benzoporphyrin derivative or chemical variants of benzoporphyrin derivative; and
activating the photosensitizers though light activation, whereby the photoactivatable nanoparticles generate reactive oxygen species and induce immunogenic cell death in the cancer cells.

10. The method of claim 9, wherein the benzoporphyrin derivative is bound to phosphatidylcholine or cholesterol.

11. The method of claim 10, wherein the benzoporphyrin derivative is bound to 20:0 lysophosphatidylcholine or 16:0 lysophosphatidylcholine.

12. The method of claim 9, wherein the lipid nanoparticles further comprise ionizable lipids.

13. The method of claim 12, wherein the ionizable lipids comprise SM-102.

14. The method of claim 9, wherein the reactive oxygen species is Type 1 reactive oxygen species.

15. The method of claim 9, wherein the photoactivatable nanoparticles localize to endoplasmic reticulum and mitochondria of the cancer cells.

16. The method of claim 9, further comprising a step of delivering additional immune checkpoint inhibitors to the subject.

17. The method of claim 16, wherein the additional immune checkpoint inhibitors comprise anti-PD-1 or anti-PD-L1.

18. The method of claim 16, wherein the additional immune checkpoint inhibitors are conjugated to or encapsulated in the lipid nanoparticles.

19. A method for designing photoactivatable nanoparticles having enhanced activity as therapeutic agents, comprising:
using data-driven statistical tool or unsupervised machine learning algorithm to evaluate properties and attributes of photoactivatable nanoparticles and produce evaluation results, wherein the properties and attributes comprise photophysical and photochemical properties;
using the evaluation results of the Principal Component Analysis to predict relationships between the properties and attributes of the various photoactivatable nanoparticles and efficiency properties of the various photoactivatable nanoparticles, wherein the efficiency properties comprise subcellular localization, type of ROS generation, and induction of immunogenic cell death markers; and
designing photoactivable nanoparticles having optimized efficiency properties and enhanced activity as therapeutic agents.

20. The method of claim 19, wherein the data-driven statistical tool or unsupervised machine learning algorithm is Principal Component Analysis.

21. The method of claim 19, wherein the properties and attributes of the photoactivatable nanoparticles comprise one or more of liposome lipid nanoparticles, solid lipid nanoparticles, unbound benzoporphyrin derivative, benzoporphyrin derivative bound to 20:0 lysophosphatidylcholinephos, benzoporphyrin derivative bound to 16:0 lysophosphatidylcholine, benzoporphyrin derivative bound to cholesterol, lipid nanoparticles comprising ionizable lipids, and administration with an additional immune checkpoint inhibitor.

22. The method of claim 19, wherein the efficiency properties comprise subcellular localization to endoplasmic reticulum and mitochondria, Type I ROS generation, and induction of immunogenic cell death.

23. A method for enhancing immune cell infiltration and reducing immunosuppression in a subject, comprising:
delivering photoactivatable nanoparticles to cancer tissue of the subject for use as therapeutic agents, wherein the photoactivatable nanoparticles comprise lipid nanoparticles and photosensitizers incorporated within the lipid nanoparticles, wherein the lipid nanoparticles are liposomes or solid lipid nanoparticles, wherein the lipid nanoparticles comprise phospholipids and lipids bound to polyethylene glycol, wherein the photosensitizers are benzoporphyrin derivative or chemical variants of benzoporphyrin derivative; and
activating the photosensitizers through light activation, whereby the photoactivatable nanoparticles generate reactive oxygen species and enhance immune cell infiltration and reduce immunosuppression in the subject.

24. The method of claim 23, further comprising a step of delivering immune cells to the cancer tissue of the subject.

25. The method of claim 23, wherein the immune cells are macrophage or natural killer (NK) cells, or wherein the immune cells are derived from adoptive cell therapy or chimeric antigen receptor cell therapy.
